(19) United States
(12) Patent Application Publication
(10) Pub. No.: US 2005/0186580 A1
(43) Pub. Date: Aug. 25, 2005

(54) Quality control method for array manufacture

(76) Inventors: Douglas J. Dellinger, Boulder, CO (US); Barry E. Boyes, Wilmington, DE (US); Gordon R. Nicol, Middletown, DE (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/785,898

(22) Filed: Feb. 23, 2004

(51) Int. Cl. C12Q 1/68

(52) U.S. Cl. 435/6

(57) ABSTRACT

A method of analyzing an array during and/or after fabrication to obtain information relating to the quality of the array manufacturing process is described. The method includes providing an array of features on a substrate, wherein each feature has one or more polynucleotides bound to the substrate. At least one of the features of the provided array is a cleavable feature. The cleavable feature has one or more polynucleotides bound to the substrate via a cleavable linker. The cleavable feature is then contacted with a matrix material, and a MALDI-MS protocol is used to obtain information about the one or more polynucleotides of the cleavable feature. This information may then be used to evaluate the manufacturing process.

### RELATED APPLICATIONS

Related subject matter is disclosed in a U.S. patent application entitled “MALDI-MS Analysis of Nucleic Acids Bound to a Surface” filed concurrently with the present application by Dellinger et al., and also in U.S. Patent Applications entitled “Method of Polynucleotide Synthesis Using Modified Support”, Ser. No. 10/652,049, filed by Dellinger et al. on Aug. 30, 2003; and “Cleavable Linker for Polynucleotide Synthesis”, Ser. No. 10/652,063, filed by Dellinger et al. on Aug. 30, 2003; all of which are incorporated herein by reference in their entireties, provided that, if a conflict in definition of terms arises, the definitions provided in the present application shall be controlling.

This invention was made with Government support under Agreement No. N39998-01-9-7068. The Government has certain rights in the invention.

### DESCRIPTION

1. Field of the Invention

The invention relates generally to the manufacture of arrays, such as polynucleotide arrays (for example, DNA arrays), which are useful in diagnostic, screening, gene expression analysis, and other applications. More particularly, the invention provides a method for performing quality control testing on the arrays.

2. Background of the Invention

Arrays such as polynucleotide arrays (for example, DNA or RNA arrays), are known and are used, for example, as diagnostic or screening tools. Polynucleotide arrays include regions of usually different sequence polynucleotides arranged in a predetermined configuration on a substrate. These regions (sometimes referenced as “features”) are positioned at respective locations (“addresses”) on the substrate. The arrays, when exposed to a sample, will exhibit an observed binding pattern. This binding pattern can be detected upon reading the array. For example all polynucleotide targets (for example, DNA) in the sample can be labeled with a suitable label (such as a fluorescent compound), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Biopolymer arrays can be fabricated by depositing previously obtained biopolymers (such as from synthesis or natural sources) onto a substrate, or by in situ synthesis methods. Methods of depositing obtained biopolymers include loading then touching a pin or capillary to a surface, such as described in U.S. Pat. No. 5,807,522 or deposition by firing from a pulse jet such as an inkjet head, such as described in PCT publications WO 95/25116 and WO 98/41531, and elsewhere. Such a deposition method can be regarded as forming each feature by one cycle of attachment (that is, there is only one cycle at each feature during which the previously obtained biopolymer is attached to the substrate). For in situ fabrication methods, multiple different reagent droplets are deposited by pulse jet or other means at a given target location in order to form the final feature (hence a probe of the feature is synthesized on the array substrate). The in situ fabrication methods include those described in U.S. Pat. No. 5,449,754 for synthesizing peptide arrays, and in U.S. Pat. No. 6,180,351 and WO 98/41531 and the references cited therein for polynucleotides, and may also use pulse jets for depositing reagents.

The in situ method for fabricating a polynucleotide array typically follows, at each of the multiple different addresses at which features are to be formed, the same conventional iterative sequence used in forming polynucleotides from nucleoside reagents on a support by means of known chemistry. This process is illustrated schematically in FIG. 1 (wherein “B” typically represents a purine or pyrimidine base, “DMT” represents dimethoxytrityl, “iPR” represents isopropyl, and “●-” represents the growing polynucleotide strand bound to the solid phase). In the first step (“deprotection”) of the four-step cycle, the 5′-O-dimethoxytrityl (DMT) group is removed from a deoxynucleoside linked to the polymer support. Step 2 (“condensation”), elongation of a growing oligodeoxynucleotide, occurs via the initial formation of a phosphite triester internucleotide bond. This reaction product is first treated with a capping agent (step 3—“capping”) designed to esterify failure sequences and cleave phosphite reaction products on the heterocyclic bases. The nascent phosphite internucleotide linkage is then oxidized to the corresponding phosphotriester (step 4—“oxidation”). The synthesis then continues with the deprotection step, removing the protecting group (“deprotection”) from the now support-bound deoxynucleoside bound to the support in the just-completed cycle, to generate a reactive site for the next cycle of these steps. The coupling can be performed by depositing drops of an activator and phosphoramidite at the specific desired feature locations for the array. A final deprotection step is provided in which nitrogenous bases and phosphate group are simultaneously deprotected by treatment with ammonium hydroxide and/or methylamine under known conditions. Capping, oxidation and deprotection can be accomplished by treating the entire substrate (“flooding”) with a layer of the appropriate reagent. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in another flooding procedure in a known manner. Conventionally, a single pulse jet or other dispenser is assigned to deposit a single monomeric unit.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643, EP 0294196, and elsewhere The phosphoramidite and phosphite triester approaches are most broadly used, but other approaches include the phosphodiester approach, the phosphotriester approach and the H-phosphonate approach. See Letsinger, R. L. et al.; J. Am. Chem. Soc. (1976) 98: 3655-61; Beaucage, S. L. et al.; Tetrahedron Lett. (1981) 22: 1859-62; and Matteucci, M. D., et al.; J. Am. Chem. Soc. (1981) 103: 3186-91.

Another method of polynucleotide synthesis has been reported whereby the oxidation and deprotection reactions are performed simultaneously using a mildly basic solution of peroxy anions (FIG. 2). See U.S. Pat. No. 6,222,030 and U.S. patent application Ser. No. 09/916,369 filed Jul. 27, 2001. See also Sierzchala, A. B., et al., J. Am. Chem. Soc. (2003) in press. For this new synthesis approach, the trityl protecting group typically used for the monomers in the traditional four-step phosphoramidite-based synthesis (FIG. 1) is not used, and further reports of the new synthesis approach have described the use of trityl group chemistry for providing a cleavable linker for surface attachment. See U.S. patent application Ser. No. 10/652,063 filed Aug. 30, 2003 by Dellinger et al.

A variety of applications in the fields of genomics and high throughput screening have fueled the demand for highly parallel, microscale synthesis of DNA and for DNA sequences attached to planar glass surfaces. An application which exemplifies this trend is DNA arrays. See Fodor, S. A., Science (1997) 277: 393-95; Lipshutz, R. J. et al.; Nature Genetics Microarray Supplement (1999) 21: 20-24. DNA may be synthesized on array surfaces using a process that includes the removal of a photolabile protecting group from the sugar using a photomasking process. McGall, G. H., et al.; J. Am. Chem. Soc. (1997) 119: 5081-90. An alternate method uses inkjet printing apparatus to deposit DNA monomer phosphoramidite reagents onto an array surface, whereby the array features are defined by addressing specific reagents at defined sites on the array surface. Hughes, T. R., et al., Nat. Biotechnol. (2001) 19(4): 342-47.

The substrates used in the manufacture of the arrays are typically functionalized to bond to the first deposited monomer. Suitable techniques for functionalizing substrates with such linking moieties are described, for example, in Southern, E. M., Maskos, U. and Elder, J. K., Genomics, 13, 1007-1017, 1992. In the case of array fabrication, different monomers and activator may be deposited at different addresses on the substrate during any one cycle so that the different features of the completed array will have different desired biopolymer sequences. One or more intermediate further steps may be required in each cycle, such as the conventional oxidation, capping and washing steps in the case of in situ fabrication of polynucleotide arrays (again, these steps may be performed in flooding procedure).

Further details of fabricating biopolymer arrays by depositing either previously obtained biopolymers or by the in situ method are disclosed in U.S. Pat. No. 6,242,266, U.S. Pat. No. 6,232,072, U.S. Pat. No. 6,180,351, and U.S. Pat. No. 6,171,797. In fabricating arrays by depositing previously obtained biopolymers or by the in situ method, typically each region on the substrate surface on which an array will be or has been formed (“array regions”) is completely exposed to one or more reagents. For example, in either method the array regions will often be exposed to one or more reagents to form a suitable layer on the surface which binds to both the substrate and biopolymer or biomonomer. In in situ fabrication the array regions will also typically be exposed to the oxidizing, deblocking, and optional capping reagents. Similarly, particularly in fabrication by depositing previously obtained biopolymers, it may be desirable to expose the array regions to a suitable blocking reagent to block locations on the surface at which there are no features from non-specifically binding to target.

In array fabrication, the quantities of polynucleotide available are usually very small and expensive. Additionally, sample quantities available for testing are usually also very small and it is therefore desirable to simultaneously test the same sample against a large number of different probes on an array. These conditions require use of arrays with large numbers of very small, closely spaced features. About 10 to 20 of such arrays can be fabricated on a rigid substrate (such as glass). Such a substrate must be manually or machine placed into a fabricating tool, and is later cut into substrate segments each of which may carry one or two arrays. Other fabrication methods require the use of flexible substrates in the form of “webbing” (e.g. a thin, flexible polymer media, such as a tape) that may be wound on reels and manipulated in a reel-to-reel fashion on equipment adapted to that purpose. See, for example, U.S. patent application Ser. No. 10/032,608 filed Oct. 18, 2001 by Lefkowitz et al.

Array fabrication typically is a demanding process. The chemical synthesis of the polynucleotides is prone to errors, and the small scales involved require precise delivery of reagents. Various methods have been used to improve the quality of the final product during the manufacture of bioarrays. One strategy described is visualizing the deposition process to reduce errors in delivery of reagent solutions to the substrate surface. See, e.g., U.S. Pat. No. 6,232,072 to Fisher. Other strategies include attempts to optimize the synthesis of the polynucleotides to the small scales involved in array fabrication. Also, arrays are typically subjected to analysis at various times during and after fabrication to ensure quality of the manufacturing process. Typical means of analysis include chromatographic or electrophoretic separations, chemical analyses, specific enzymatic cleavage reactions, and other means.

It would also be desirable to have a convenient way to analyze the arrays during and/or after fabrication to obtain information relating to the quality of the array manufacturing process.

### SUMMARY OF THE INVENTION

We have now developed such a convenient method of analyzing an array during and/or after fabrication to obtain information relating to the quality of the array manufacturing process. The method includes providing an array including many features on a substrate, wherein each feature has one or more polynucleotides bound to the substrate. At least one of the features of the provided array is a cleavable feature, which has one or more polynucleotides bound to the substrate via a cleavable linker. The cleavable feature is then contacted with a matrix material and the substrate is placed in operable association with a MALDI source apparatus. The one or more polynucleotides of the cleavable feature is analyzed using MALDI-MS to obtain information about the one or more polynucleotides of the cleavable feature. This information may then be used to evaluate the manufacturing process, e.g. to provide feedback to the manufacturing process or to provide a quality control measurement for the provided array or for other arrays contemporaneously manufactured with the provided array.

Arrays having polynucleotides bound to a substrate via a cleavable linker, cleavable linkers, and methods of evaluating an array manufacturing process are further described herein. Additional objects, advantages, and novel features of this invention shall be set forth in part in the descriptions and examples that follow and in part will become apparent to those skilled in the art upon examination of the following specifications or may be learned by the practice of the invention. The objects and advantages of the invention may be realized and attained by means of the materials and methods particularly pointed out in the appended claims.

### BRIEF DESCRIPTION OF THE DRAWINGS

These and other features of the invention will be understood from the description of representative embodiments of the method herein and the disclosure of illustrative materials for carrying out the method, taken together with the Figures, wherein

FIG. 1 schematically illustrates prior art synthesis of polynucleotides.

FIG. 2 depicts a prior art synthesis scheme for synthesizing polynucleotides, the synthesis scheme employing a two step synthesis cycle, including a coupling step and a simultaneous deprotection and oxidation step.

FIG. 3 illustrates an array substrate such as may be provided in an embodiment of the present invention.

FIG. 4 illustrates a multi-array substrate such as may be provided in an embodiment of the present invention.

FIG. 5 shows an embodiment in accordance with the present invention, in which a polynucleotide is release from a substrate in a MALDI-MS analysis method.

FIG. 6 shows an embodiment in accordance with the present invention, in which a phosphoramidite is coupled to a substrate.

FIG. 7 give shows mass spectra resulting from a MALDI-MS analysis, described herein.

FIG. 8 shows mass spectra prepared in accordance with the present invention.

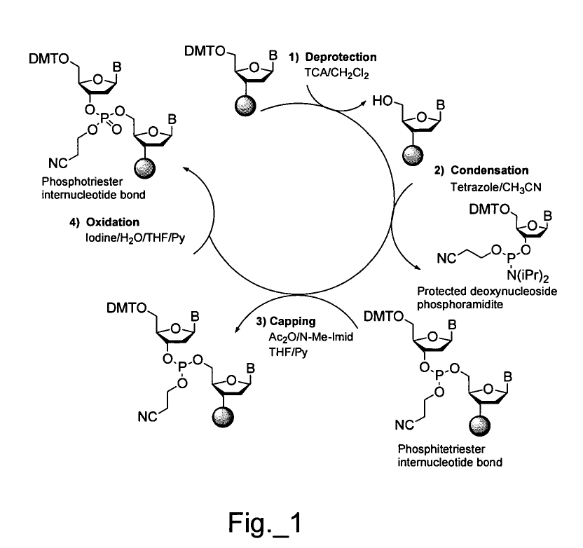

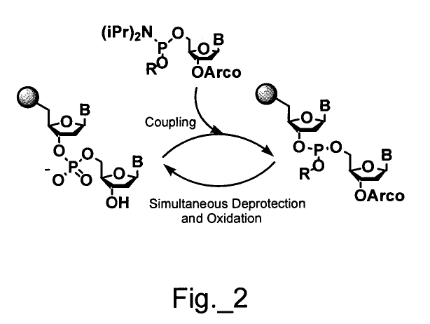

To facilitate understanding, identical reference numerals/designations have been used, where practical, to designate corresponding elements that are common to the Figures. Figure components are not drawn to scale.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different sequence where this is logically possible. However, the sequence described below is preferred.

It must be noted that, as used in the specification and the appended claims, the singular forms “a,” “an” and “the” include plural referents unless the context clearly dictates otherwise. Thus, for example, reference to “a solid support” includes a plurality of insoluble supports. Likewise, reference to “a polynucleotide” includes embodiments having a plurality of polynucleotides. Similarly, reference to “a substituent”, as in a compound substituted with “a substituent”, includes the possibility of substitution with more than one substituent, wherein the substituents may be the same or different. In this specification and in the claims that follow, reference will be made to a number of terms that shall be defined to have the following meanings unless a contrary intention is apparent.

An “array”, unless a contrary intention appears, includes any one, two or three dimensional arrangement of addressable regions bearing a particular chemical moiety or moieties (for example, polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other). While probes and targets of the present invention will typically be single-stranded, this is not essential. The probes are typically covalently bound to a substrate, and as used herein, the term “cleavable feature” indicates a region of an array having a probe bound to the substrate via a cleavable linker according to the present invention. In comparison, as used herein, the term “stable feature” indicates a region of an array in which the probe is bound to the substrate via a linking group that is NOT a cleavable linker as disclosed herein, e.g. the probe is bound via a linker moiety that is not appreciably cleaved (i.e. is stable) under conditions typically encountered in manufacture, analysis, or use of the array. An “array layout” refers to one or more characteristics of the array, such as feature positioning, feature size, and some indication of a moiety at a given location. “Hybridizing” and “binding”, with respect to polynucleotides, are used interchangeably.

A “nucleotide” refers to a sub-unit of a nucleic acid (whether DNA or RNA or analogue thereof) which includes a phosphate group, a sugar group and a heterocyclic base, as well as analogs of such sub-units. A “nucleoside” references a nucleic acid subunit including a sugar group and a heterocyclic base. A “nucleoside moiety” refers to a portion of a molecule having a sugar group and a heterocyclic base (as in a nucleoside); the molecule of which the nucleoside moiety is a portion may be, e.g. a polynucleotide, oligonucleotide, or nucleoside phosphoramidite. A “nucleotide monomer” refers to a molecule which is not incorporated in a larger oligo- or poly-nucleotide chain and which corresponds to a single nucleotide sub-unit; nucleotide monomers may also have activating or protecting groups, if such groups are necessary for the intended use of the nucleotide monomer. A “polynucleotide intermediate” references a molecule occurring between steps in chemical synthesis of a polynucleotide, where the polynucleotide intermediate is subjected to further reactions to get the intended final product, e.g. a phosphite intermediate which is oxidized to a phosphate in a later step in the synthesis, or a protected polynucleotide which is then deprotected. An “oligonucleotide” generally refers to a nucleotide multimer of about 2 to 200 nucleotides in length, while a “polynucleotide” includes a nucleotide multimer having at least two nucleotides and up to several thousand (e.g. 5000, or 10,000) nucleotides in length. It will be appreciated that, as used herein, the terms “nucleoside”, “nucleoside moiety” and “nucleotide” will include those moieties which contain not only the naturally occurring purine and pyrimidine bases, e.g., adenine (A), thymine (T), cytosine (C), guanine (G), or uracil (U), but also modified purine and pyrimidine bases and other heterocyclic bases which have been modified (these moieties are sometimes referred to herein, collectively, as “purine and pyrimidine bases and analogs thereof”). Such modifications include, e.g., methylated purines or pyrimidines, acylated purines or pyrimidines, and the like, or the addition of a protecting group such as acetyl, difluoroacetyl, trifluoroacetyl, isobutyryl, benzoyl, or the like. The purine or pyrimidine base may also be an analog of the foregoing; suitable analogs will be known to those skilled in the art and are described in the pertinent texts and literature. Common analogs include, but are not limited to, 1-methyladenine, 2 methyladenine, N6-methyladenine, N6-isopentyladenine, 2-methylthio-N-6-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine.

The term “alkyl” as used herein, unless otherwise specified, refers to a saturated straight chain, branched or cyclic hydrocarbon group of 1 to 24, typically 1-12, carbon atoms, such as methyl, ethyl, n-propyl, isopropyl, n-butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. The term “lower alkyl” intends an alkyl group of one to six carbon atoms, and includes, for example, methyl, ethyl, n-propyl, isopropyl, n-butyl, isobutyl, t-butyl, pentyl, cyclopentyl, isopentyl, neopentyl, hexyl, isohexyl, cyclohexyl, 3-methylpentyl, 2,2-dimethylbutyl, and 2,3-dimethylbutyl. The term “cycloalkyl” refers to cyclic alkyl groups such as cyclopropyl, cyclobutyl, cyclopentyl, cyclohexyl, cycloheptyl and cyclooctyl.

The term “modified alkyl” refers to an alkyl group having from one to twenty-four carbon atoms, and further having additional groups, such as one or more linkages selected from ether-, thio-, amino-, phospho-, oxo-, ester-, and amido-, and/or being substituted with one or more additional groups including lower alkyl, aryl, alkoxy, thioalkyl, hydroxyl, amino, sulfonyl, thio, mercapto, imino, halo, cyano, nitro, nitroso, azido, carboxy, sulfide, sulfone, sulfoxy, phosphoryl, silyl, silyloxy, and boronyl. The term “modified lower alkyl” refers to a group having from one to six carbon atoms and further having additional groups, such as one or more linkages selected from ether-, thio-, amino-, phospho-, keto-, ester- and amido-, and/or being substituted with one or more groups including lower alkyl; aryl, alkoxy, thioalkyl, hydroxyl, amino, sulfonyl, thio, mercapto, imino, halo, cyano, nitro, nitroso, azido, carboxy, sulfide, sulfone, sulfoxy, phosphoryl, silyl, silyloxy, and boronyl. The term “alkoxy” as used herein refers to a substituent —O—R wherein R is alkyl as defined above. The term “lower alkoxy” refers to such a group wherein R is lower alkyl. The term “thioalkyl” as used herein refers to a substituent —S—R wherein R is alkyl as defined above.

The term “alkenyl” as used herein, unless otherwise specified, refers to a branched, unbranched or cyclic (e.g. in the case of C5 and C6) hydrocarbon group of 2 to 24, typically 2 to 12, carbon atoms containing at least one double bond, such as ethenyl, vinyl, allyl, octenyl, decenyl, and the like. The term “lower alkenyl” intends an alkenyl group of two to six carbon atoms, and specifically includes vinyl and allyl. The term “cycloalkenyl” refers to cyclic alkenyl groups.

The term “alkynyl” as used herein, unless otherwise specified, refers to a branched or unbranched hydrocarbon group of 2 to 24, typically 2 to 12, carbon atoms containing at least one triple bond, such as acetylenyl, ethynyl, n-propynyl, isopropynyl, n-butynyl, isobutynyl, t-butynyl, octynyl, decynyl and the like. The term “lower alkynyl” intends an alkynyl group of two to six carbon atoms, and includes, for example, acetylenyl and propynyl, and the term “cycloalkynyl” refers to cyclic alkynyl groups.

The term “aryl” as used herein refers to an aromatic species containing 1 to 5 aromatic rings, either fused or linked, and either unsubstituted or substituted with one or more substituents typically selected from the group consisting of lower alkyl, aryl, aralkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, and boronyl; and lower alkyl substituted with one or more groups selected from lower alkyl, alkoxy, thioalkyl, hydroxyl thio, mercapto, amino, imino, halo, cyano, nitro, nitroso, azido, carboxy, sulfide, sulfone, sulfoxy, phosphoryl, silyl, silyloxy, and boronyl. Typical aryl groups contain 1 to 3 fused aromatic rings, and more typical aryl groups contain 1 aromatic ring or 2 fused aromatic rings. Aromatic groups herein may or may not be heterocyclic. The term “aralkyl” intends a moiety containing both alkyl and aryl species, typically containing less than about 24 carbon atoms, and more typically less than about 12 carbon atoms in the alkyl segment of the moiety, and typically containing 1 to 5 aromatic rings. The term “aralkyl” will usually be used to refer to aryl-substituted alkyl groups. The term “aralkylene” will be used in a similar manner to refer to moieties containing both alkylene and aryl species, typically containing less than about 24 carbon atoms in the alkylene portion and 1 to 5 aromatic rings in the aryl portion, and typically aryl-substituted alkylene. Exemplary aralkyl groups have the structure —CH2)j-Ar wherein j is an integer in the range of 1 to 24, more typically 1 to 6, and Ar is a monocyclic aryl moiety.

The term “heterocyclic” refers to a five- or six-membered monocyclic structure or to an eight- to eleven-membered bicyclic structure which is either saturated or unsaturated. The heterocyclic groups herein may be aliphatic or aromatic. Each heterocyclic group consists of carbon atoms and from one to four heteroatoms selected from the group consisting of nitrogen, oxygen and sulfur. As used herein, the term “nitrogen heteroatoms” includes any oxidized form of nitrogen and the quaternized form of nitrogen. The term “sulfur heteroatoms” includes any oxidized form of sulfur. Examples of heterocyclic groups include purine, pyrimidine, piperidinyl, morpholinyl and pyrrolidinyl. “Heterocyclic base” refers to any natural or non-natural heterocyclic moiety that can participate in base pairing or base stacking interaction on an oligonucleotide strand.

“Moiety” and “group” are used interchangeably herein to refer to a portion of a molecule, typically having a particular functional or structural feature, e.g. a linking group (a portion of a molecule connecting two other portions of the molecule), or an ethyl moiety (a portion of a molecule with a structure closely related to ethane). A “triaryl methyl linker group” as used herein references a triaryl methyl group having one or more substituents on the aromatic rings of the triaryl methyl group, wherein the triaryl methyl group is bonded to two other moieties such that the two other moieties are linked via the triaryl methyl group. An “intermediate linking group” references any linking group adjacent to the triaryl methyl linker group and bound to the triaryl methyl linker group. “Linkage” as used herein refers to a first moiety bonded to two other moieties, wherein the two other moieties are linked via the first moiety. Typical linkages include ether (—O—), oxo (—C(O)—), amino (—NH—), amido (—N—C(O), thio (—S—), phospho (—P—), ester (—O—C(O)—).

“Bound” may be used herein to indicate direct or indirect attachment. In the context of chemical structures, “bound” (or “bonded”) may refer to the existence of a chemical bond directly joining two moieties or indirectly joining two moieties (e.g. via a linking group). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and also embodiments where the attachment is indirect. Depending on the context, “connected”, “linked”, or other like term indicates that two groups are bound to each other, wherein the attachment may be direct or indirect.

“Functionalized” references a process whereby a material is modified to have a specific moiety bound to the material, e.g. a molecule or substrate is modified to have the specific moiety; the material (e.g. molecule or substrate) that has been so modified is referred to as a functionalized material (e.g. functionalized molecule or functionalized substrate).

The term “halo” or “halogen” is used in its conventional sense to refer to a chloro, bromo, fluoro or iodo substituent.

By “protecting group” as used herein is meant a species which prevents a portion of a molecule from undergoing a specific chemical reaction, but which is removable from the molecule following completion of that reaction. This is in contrast to a “capping group,” which permanently binds to a segment of a molecule to prevent any further chemical transformation of that segment. A “hydroxyl protecting group” refers to a protecting group where the protected group is a hydroxyl. “Reactive site hydroxyl” references a hydroxyl group capable of reacting with an activated nucleotide monomer to result in an internucleotide bond being formed. In typical embodiments, the reactive site hydroxyl is the terminal 5′-hydroxyl during 3′-5′ polynucleotide synthesis and is the 3′-hydroxyl during 5′-3′ polynucleotide synthesis. An “acid labile protected hydroxyl” is a hydroxyl group protected by a protecting group that can be removed by acidic conditions. Similarly, an “acid stabile protected hydroxyl” is a hydroxyl group protected by a protecting group that is not removed (is stabile) under acidic conditions. An “acid labile linking group” is a linking group that releases a linked group under acidic conditions. A “cleavable linker” is a linking group that functions to temporarily attach a polynucleotide to a substrate, and which releases the polynucleotide from the substrate under appropriate conditions. This is in contrast to linking groups that bind a polynucleotide probe to the substrate via a linker moiety that is not appreciably cleaved (i.e. is stable) under conditions typically encountered in manufacture, analysis, or use of the array. In certain embodiments, the cleavable linker in accordance with the invention is an acid labile linking group.

A trityl group is a triphenyl methyl group, in which one or more of the phenyl groups of the triphenyl methyl group is optionally substituted. A “substituted trityl group” or a “substituted triphenyl methyl group” is a triphenyl methyl group on which at least one of the hydrogens of the phenyl groups of the triphenyl methyl group is replaced by a substituent.

The term “substituted” as used to describe chemical structures, groups, or moieties, refers to the structure, group, or moiety comprising one or more substituents. As used herein, in cases in which a first group is “substituted with” a second group, the second group is attached to the first group whereby a moiety of the first group (typically a hydrogen) is replaced by the second group.

“Substituent” references a group that replaces another group in a chemical structure. Typical substituents include nonhydrogen atoms (e.g. halogens), functional groups (such as, but not limited to amino, sulfhydryl, carbonyl, hydroxyl, alkoxy, carboxyl, silyl, silyloxy, phosphate and the like), hydrocarbyl groups, and hydrocarbyl groups substituted with one or more heteroatoms. Exemplary substituents include alkyl, lower alkyl, aryl, aralkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, boronyl, and modified lower alkyl.

A “group” includes both substituted and unsubstituted forms. Typical substituents include one or more lower alkyl, modified alkyl, any halogen, hydroxy, or aryl. Any substituents are typically chosen so as not to substantially adversely affect reaction yield (for example, not lower it by more than 20% (or 10%, or 5% or 1%) of the yield otherwise obtained without a particular substituent or substituent combination).

Hyphens, or dashes, are used at various points throughout this specification to indicate attachment, e.g. where two named groups are immediately adjacent a dash in the text, this indicates the two named groups are attached to each other. Similarly, a series of named groups with dashes between each of the named groups in the text indicates the named groups are attached to each other in the order shown. Also, a single named group adjacent a dash in the text indicates the named group is typically attached to some other, unnamed group. In some embodiments, the attachment indicated by a dash may be, e.g. a covalent bond between the adjacent named groups. In some other embodiments, the dash may indicate indirect attachment, i.e. with intervening groups between the named groups. At various points throughout the specification a group may be set forth in the text with or without an adjacent dash, (e.g. amido or amido-, further e.g. Trl or Trl-, yet further e.g. Cgp, Cgp- or -Cgp-) where the context indicates the group is intended to be (or has the potential to be) bound to another group; in such cases, the identity of the group is denoted by the group name (whether or not there is an adjacent dash in the text). Note that where context indicates, a single group may be attached to more than one other group (e.g. the triaryl methyl linker group, herein; further e.g. where a linkage is intended, such as linking groups).

The term “MALDI-MS” references matrix assisted laser desorption/ionization mass spectrometry, which entails methods of mass spectrometric analysis which use a laser as a means to desorb, volatize, and ionize an analyte. In MALDI-MS methods, the analyte is contacted with a matrix material to prepare the analyte for analysis. The matrix material absorbs energy from the laser and transfers the energy to the analyte to desorb, volatize, and ionize the analyte, thereby producing ions from the analyte that are then analyzed in the mass spectrometer to yield information about the analyte. A “MALDI sample plate” is a device that, when disposed in an operable relationship with a laser desorption ionization source of a MALDI mass spectrometer, can be used to deliver ions derived from an analyte on the device to the mass spectrometer for analysis to obtain information about the analyte. In other words, the term “MALDI sample plate” refers to a device that is removably insertable into a MALDI mass spectrometer and contains a substrate having a surface for presenting analytes for detection by the mass spectrometer. Other references may refer to a MALDI sample plate, as used herein, as a “target” or a “probe”.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present. At various points herein, a moiety may be described as being present zero or more times: this is equivalent to the moiety being optional and includes embodiments in which the moiety is present and embodiments in which the moiety is not present. If the optional moiety is not present (is present in the structure zero times), adjacent groups described as linked by the optional moiety are linked to each other directly. Similarly, a moiety may be described as being either (1) a group linking two adjacent groups, or (2) a bond linking the two adjacent groups: this is equivalent to the moiety being optional and includes embodiments in which the moiety is present and embodiments in which the moiety is not present. If the optional moiety is not present (is present in the structure zero times), adjacent groups described as linked by the optional moiety are linked to each other directly.

Accordingly, the method of the present invention finds utility in performing quality control assays of array manufacturing processes. A method in accordance with the present invention provides for analyzing an array during and/or after fabrication to provide quality control for the array manufacturing process. In this regard, “quality control” refers to the measurement of observable characteristics of the array manufacturing process, including the products of that process (e.g. the arrays), to determine whether manufacturing parameters and/or product characteristics are within appropriate ranges. The manufacturing parameters may be adjusted as necessary to maintain the parameters within the appropriate ranges. Data obtained as a result of the measurement of observable characteristic in a quality control assay is referenced as a “quality control measurement”.

The method includes providing an array including a plurality of features on a substrate, wherein each feature has one or more polynucleotides bound to the substrate. At least one of the features of the provided array is a cleavable feature, which has one or more polynucleotides bound to the substrate via a cleavable linker. The cleavable feature is then contacted with a matrix material and the substrate is placed in operable association with a MALDI source apparatus. The one or more polynucleotides of the cleavable feature is analyzed using MALDI-MS to obtain information about the polynucleotide. This information may then be used to evaluate the manufacturing process, e.g. to provide feedback to the manufacturing process or to provide a quality control measurement for the provided array or for other arrays contemporaneously manufactured with the provided array. Providing a quality control measurement includes embodiments in which the quality control measurement is an actual measurement from an assay performed on the array (e.g. the array provided in the method of the present invention). Providing a quality control measurement also includes embodiments in which the quality control measurement is an inferred measurement. For example, in an embodiment in which an actual quality control measurement has been obtained from an assay performed on an array provided in the method of the present invention, other arrays contemporaneously manufactured with the provided array may have a corresponding quality control measurement inferred. The inferred quality control measurement may include a determination that a production lot meets specific quality criteria based on information obtained by subjecting one or more arrays from the production lot to analysis in accordance with the method of the present invention.

An array provided in accordance with the method of the invention including a plurality of features on a substrate, wherein each feature has one or more polynucleotides bound to the substrate. Each feature occupies a specific site, or “address” on the array. The array typically has at least about 10 features, typically at least about 40 features, typically at least about 100 features, and may have up to about 1000 features, typically up to about 4000 features, or even more. The array may be provided in finished form, i.e. having completed the manufacturing process, or may be incomplete, i.e. removed from the manufacturing process at an intermediate step of the manufacturing process (prior to completion).

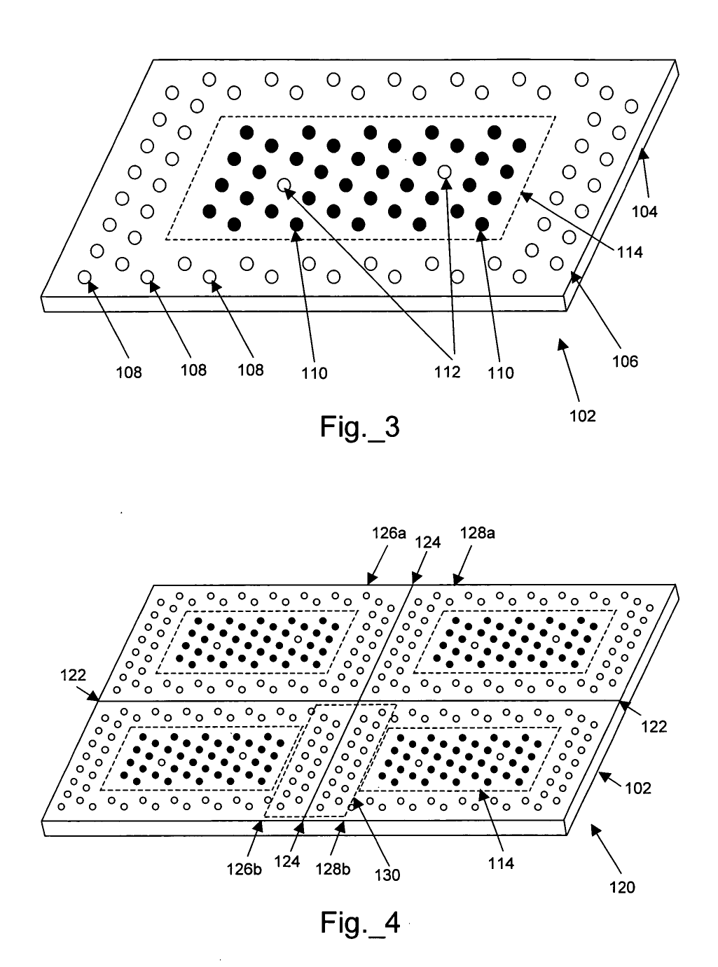

Referring now to FIG. 3, an array 102 provided in accordance with the method of the invention is illustrated. The array 102 comprises a substrate 104 having a surface 106 and a plurality of features 108, 110, 112 on the surface 106. In the illustrated embodiment, each feature represented by the open circles 108 is a cleavable feature (as defined herein) and has one or more polynucleotides bound to the surface 106 of the substrate 104 via a cleavable linking group capable of covalently binding the one or more polynucleotides to the substrate. In contrast, each feature represented by the filled circles 110 is a stable feature (as defined herein) having one or more polynucleotides bound to the substrate via a linking group that is stable. The stable features 110 in the illustrated embodiment are disposed in a central area 114 of the surface 106. In certain embodiments, cleavable features may be present at regular or irregular intervals in the central area. In certain embodiments, the central area 114 corresponds to the finished array product; in such embodiments, the surrounding areas of the substrate are typically separated from the central area and submitted to analysis in accordance with the method of the present invention.

Arrays such as those illustrated in FIG. 3 are typically obtained by first producing a multi-array substrate to take advantage of economies of scale and automation in the manufacturing process. A multi-array substrate 120 is illustrated in FIG. 4; the multi-array substrate includes four arrays such as are illustrated in FIG. 3. The multi-array substrate 120 may form a part of a larger substrate having many arrays, but only four are shown in the figure. The multi-array substrate will typically be cut or otherwise divided along the lines indicated by the arrows 122 and 124 to result in four arrays such as are illustrated in FIG. 3. However, in an alternate embodiment, the multi-array substrate is cut or otherwise divided along the line indicated by arrows 122 and is further divided along a line extending from arrow 126a to arrow 126b, and is also further divided along a lone extending from arrow 128a to arrow 128b, to result in an array of cleavable features (i.e. the cleavable features disposed between the lines defined by arrows 126a,b and 128a,b, e.g. corresponding to the area enclosed in the dashed line designated by the arrow 130) separate from the array designated by the central area 114. The array of cleavable features is analyzed in accordance with the method of the present invention, and gives a useful measure of the characteristics of the other arrays being fabricated contemporaneously in the manufacturing process, e.g. on the same multi-array substrate, further e.g. in the same production lot.

In still other embodiments, the areas surrounding the central areas are omitted, and the central areas provide the substrate to be analyzed in accordance with the method of the present invention. In such embodiments, the cleavable features may be disposed at regular or irregular intervals on the surface among the stable features. In some embodiments, a substantial percentage of the arrays fabricated on a multi-array substrate include only stable features, and the remaining small percentage of the arrays fabricated on the same multi-array substrate include only cleavable features (or in some embodiments may include a combination of cleavable and stable features). The substantial percentage is typically at least 60 number percent, or more typically at least 70 number percent, or more typically at least 80 number percent, or more typically at least 90 number percent, or still more typically at least about 98 number percent. Typically the small percentage of arrays with cleavable features is less than about 40 number percent, more typically less than about 30 number percent, more typically less than about 20 number percent, more typically less than about 10 number percent, or typically less than about 2 number percent.

The cleavable features on the arrays provide a convenient means of analyzing the substrates to test the manufacturing process. It will be appreciated that the description of FIG. 3 and FIG. 4 includes elements that may be optional or may be assorted in any other suitable combinations, such as will be apparent to those of skill in the art. For example, the format of the arrays on the multi-array substrates may vary depending on design and intended use. In particular embodiments, the multi-array substrate is divided to provide substrates having a single array on a substrate; in other embodiments, two, three, or four, or more arrays on a single substrate are provided in accordance with the method of the invention. Still other embodiments will be readily apparent to those of skill in the art given the disclosure herein and are intended to be encompassed in the present invention.

For example, the features of the array may be arranged in any desired pattern, e.g. organized rows and columns of features (for example, a grid of features across the substrate surface), a series of curvilinear rows across the substrate surface (for example, a series of concentric circles or semi-circles of features), and the like. In embodiments where very small feature sizes are desired, the density of features on the substrate may range from at least about ten features per square centimeter, or typically at least about 35 features per square centimeter, or more typically at least about 100 features per square centimeter, and up to about 1000 features per square centimeter, or typically up to about 10,000 features per square centimeter. Therefore, one embodiment of the invention provides an array having features that may have widths (that is, diameter, for a round spot) in the range from a minimum of about 10 micrometers to a maximum of about 1.0 cm. Interfeature areas will typically (but not essentially) be present which do not carry any polynucleotide. It will be appreciated though, that the interfeature areas could be of various sizes and configurations.

The substrate may take any of a variety of configurations ranging from simple to complex. Thus, the substrate could have generally planar form, as for example a slide or plate configuration, such as a rectangular- or square- or disc-shape. In many embodiments, the substrate will be shaped generally as a rectangular solid, having a length in the range about 4 mm to 400 mm, usually about 4 mm to 150 mm, more usually about 4 mm to 125 mm; a width in the range about 4 mm to 400 mm, usually about 4 mm to 120 mm and more usually about 4 mm to 80 mm; and a thickness in the range about 0.01 mm to 5.0 mm, usually from about 0.1 mm to 2 mm and more usually from about 0.2 to 1 mm. The substrate surface onto which the polynucleotides are bound may be smooth or substantially planar, or have irregularities, such as depressions or elevations. The configuration of the array may be selected according to manufacturing, handling, and use considerations.

In one embodiment, about 10 to 100 of such arrays can be fabricated on a single multi-array substrate (such as glass). In such embodiment, after the substrate has the polynucleotides on its surface, the substrate may be cut into substrate segments, each of which may carry one or two arrays. It will also be appreciated that there need not be any space separating arrays from one another. Where a pattern of arrays is desired, any of a variety of geometries may be constructed, including for example, organized rows and columns of arrays (for example, a grid of arrays, across the substrate surface), a series of curvilinear rows across the substrate surface (for example, a series of concentric circles or semi-circles of arrays), and the like.

Typical substrate materials provide physical support for the deposited material and endure the conditions of the deposition process and of any subsequent treatment or handling or processing that may be encountered in the use of the particular array. Suitable substrates may have a variety of forms and compositions and may derive from naturally occurring materials, naturally occurring materials that have been synthetically modified, or synthetic materials. Examples of suitable substrate materials include, but are not limited to, nitrocellulose, glasses, silicas, teflons, plastics, and metals (for example, gold, platinum, and the like), and combinations thereof. Suitable substrate materials also include polymeric materials, including plastics, for example, polytetrafluoroethylene, polypropylene, polystyrene, polycarbonate, polyvinyl alcohol, copolymers of hydroxyethyl methacrylate and methyl methacrylate, and the like, and combinations thereof.

The substrate surface may optionally exhibit surface modifications over a portion or over all of the surface with one or more different layers of compounds that serve to modify the properties of the surface in a desirable manner. Such modifications include: inorganic and organic layers such as metals, metal oxides, conformal silica or glass coatings, polymers, small organic molecules, hydrophobic or hydrophilic surface treatments, and the like.

Any suitable manufacturing process capable of providing an array in accordance with the method of the invention may be employed. The manufacturing process typically includes such steps as obtaining and preparing a substrate to receive and bind to one or more polynucleotides, contacting the substrate with the one or more polynucleotides, passivating the array after the polynucleotides are bound to the surface of the substrate, and packaging the array substrate. Typical examples of these steps are known in the art, or modified methods should be apparent to those of skill in the art given the disclosure herein. The manufacturing method is typically performed on a scale to mass produce arrays, producing on the order of tens, hundreds, or thousands of arrays in a given production lot. Arrrays “contemporaneously manufactured” are fabricated at about the same time (e.g. about the same hour, same day, or same week) in the manufacturing process, and are fabricated using substantially the same facilities and equipment. For example, arrays that are contemporaneously manufactured may have been produced as part of the same multi-array substrate. As another example, arrays that are contemporaneously manufactured may have been produced as part of the same production lot.

The array is typically fabricated in a manufacturing process that includes applying a polynucleotide (or nucleotide monomers for an in situ synthesis process) and/or other reagents to the surface of the substrate by spotting, using pipettes, pins, inkjets, or the like. Methods of depositing materials onto a planar substrate include loading and then touching a pin or capillary to a surface, such as described in U.S. Pat. No. 5,807,522 to Brown et al. U.S. Pat. No. 6,110,426 to Shalon, et al. describes a method of dispensing a known volume of a reagent at each selected array position, by tapping a capillary dispenser on the substrate under conditions effective to draw a defined volume of liquid onto the substrate. Another method employs an array of pins dipped into corresponding wells, e.g., the 96 wells of a microtitre plate, for transferring an array of droplets to a substrate, such as a porous membrane. One such array of pins is designed to spot a membrane in a staggered fashion, for creating an array of 9216 spots in a 22 by 22 cm area (Lehrach, et al., “Hybrididization Fingerprinting in Genome Mapping and Sequencing,” in Genome Analysis, Vol. 1, pp. 39-81 (1990, Davies and Tilgham, Eds., Cold Spring Harbor Press)). A different method has been described which uses a vacuum manifold to transfer a plurality, e.g., 96, of aqueous samples of DNA from 3 millimeter diameter wells to a porous membrane for making ordered arrays of DNA on a porous membrane, i.e. a “dot blot” approach. Still other methods and apparatus for fabrication of polynucleotide arrays are described in, e.g. U.S. Pat. No. 6,242,266 to Schleiffer et al., which describes a fluid dispensing head for dispensing droplets onto a substrate, and methods of positioning the head in relation to the substrate. Other methods include those disclosed by U.S. Pat. No. 6,180,351 to Cattell; U.S. Pat. No. 6,171,797 to Perbost; Gamble, et al., WO97/44134; Gamble, et al., WO98/10858; Baldeschwieler, et al., WO95/25116; and the like.

Ink jet technology may be used in the manufacturing process to spot biomolecules and/or other reagents on a surface, for example, using a pulse jet such as an inkjet type head to deposit a droplet of reagent solution for each feature. Such a technique has been described, for example, in PCT publications WO 89/10977, WO 95/25116 and WO 98/41531, and elsewhere. In such methods, the head has at least one jet which can dispense droplets of a fluid onto a substrate. Multiple fluid droplets (where the fluid comprises the biomolecule) are dispensed from the jet so as to form an array of droplets on the substrate (this formed array may or may not be the same as the final desired array since, for example, multiple heads can be used to form the final array and multiple passes of the head(s) may be required to complete the array).

A number of other known methods are available and may be used in the array manufacturing process for depositing the biomolecules on the substrate surface. It should be specifically understood that, in addition to inkjet methods, other methods can also be used to deposit biomolecules on the substrate surface, including those such as described in U.S. Pat. No. 5,807,522, or apparatus which may employ photolithographic techniques for forming arrays of moieties, such as described in U.S. Pat. No. 5,143,854 and U.S. Pat. No. 5,405,783, or any other suitable apparatus which can be used for fabricating arrays of moieties. For example, robotic devices for precisely depositing volumes of solutions onto discrete locations of a support surface, i.e. arrayers, are commercially available from a number of vendors, including: Genetic Microsystems; Cartesian Technologies; Beecher Instruments; Genomic Solutions; and BioRobotics. For further methods, see U.S. Pat. No. 5,143,854 to Pirrung et al.; Fodor et al., Science 251:767-773 (1991); Southern, et al. Genomics 13:1008-1017 (1992); PCT patent publications WO 90/15070 and 92/10092; U.S. Pat. No. 4,877,745 to Hayes et al.; U.S. Pat. No. 5,338,688 to Deeg et al.; U.S. Pat. No. 5,474,796 to Brennan; U.S. Pat. No. 5,449,754 to Nishioka; U.S. Pat. No. 5,658,802 to Hayes et al.; and U.S. Pat. No. 5,700,637 to Southern. Another strategy for forming bioarrays is discussed in U.S. Pat. No. 5,744,305 to Fodor, et al. and involves solid phase chemistry, photolabile protecting groups and photolithography. Still other patents and patent applications describing arrays of biopolymeric compounds and methods for their fabrication include: U.S. Pat. Nos. 5,242,974; 5,384,261; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,695; 5,624,711; 5,639,603; 5,658,734; WO 93/17126; WO 95/11995; WO 95/35505; EP 742 287; and EP 799 897. Also of interest are WO 97/14706 and WO 98/30575. Modifications of these known methods within the capabilities of a skilled practitioner in the art as well as other methods known to those of skill in the art may be employed.

After an array having at least one cleavable feature is provided in accordance with the method of the invention, the cleavable feature is contacted with a matrix material. The array is then placed in operable association with a MALDI source apparatus. The one or more polynucleotides of the cleavable feature is analyzed using MALDI-MS to obtain information about the polynucleotide. This information may then be used to evaluate the manufacturing process, e.g. to provide feedback to the manufacturing process or to provide a quality control measurement for the provided array or for other arrays contemporaneously manufactured with the provided array. As an example, the feedback provided may allow the adjustment of manufacturing parameters in response to the information. As a further example, the quality control measurement provided may allow for detection and elimination of sub-quality arrays, thereby allowing more consistency between production lots and within production lots of arrays.

The method of the present invention includes providing an array, wherein at least one of the features of the provided array is a cleavable feature, which has one or more polynucleotides bound to the substrate of the array via a cleavable linker. The cleavable linker serves to release the one or more bound polynucleotides from the substrate to allow analysis of polynucleotides in accordance with the present invention. The cleavable linker may be any linking group that is selectively cleaved under the appropriate conditions (depending on the identity of the cleavable linker). Typically the cleavable linker is an acid labile linking group. In particular embodiments, the cleavable linker is cleaved when the array is prepared for analysis by MALDI-MS and/or upon exposing the matrix material to laser radiation during MALDI-MS.

In the method of the present invention, the array may be obtained from a manufacturing process including a step of binding polynucleotides to an array substrate. In an embodiment, the array includes a cleavable feature that has a polynucleotide bound to the substrate via a triaryl methyl linker group, the triaryl methyl linker group covalently linking the substrate to the polynucleotide. The invention provides a method of analyzing the array using matrix assisted laser desorption/ionization mass spectrometry (MALDI-MS). In an embodiment, the cleavable feature may be represented as having the structure (I)
●-Cgp-Trl-Cgp′-Pnt (I)
wherein the groups are defined as follows:
●- is a substrate,
Trl is a triaryl methyl linker group having three aryl groups, each bound to a central methyl carbon, at least one of said three aryl groups having one or more substituents,
Cgp is a linking group linking the substrate and the triaryl methyl linker group, or is a bond linking the substrate and the triaryl methyl linker group,
Pnt is a polynucleotide, and
Cgp′ is a linking group linking the polynucleotide and the triaryl methyl linker group, or is a bond linking the polynucleotide and the triaryl methyl linker group.

The cleavable feature having the structure (I) is then contacted with a matrix material and subjected to analysis by MALDI-MS. During the MALDI-MS analysis, laser radiation is directed at the matrix material, thereby exciting the matrix material and releasing the polynucleotide from the substrate. Ions generated as a result of this excitation and release process are then analyzed to provide information about the polynucleotide. This information may then be used to evaluate the manufacturing process, e.g. to provide feedback to the manufacturing process or to provide a quality control measurement for the provided array or for other arrays contemporaneously manufactured with the provided array.

In particular embodiments, the method includes providing an array having a cleavable feature, the cleavable feature having a polynucleotide bound to a substrate via a linker moiety having a triaryl methyl linker group. The polynucleotide bound to the substrate is then contacted with a matrix material and analyzed by MALDI-MS. During the MALDI-MS analysis, laser radiation is directed at the matrix material, thereby exciting the matrix material and causing cleavage of the linker moiety. Ions generated as a result of this excitation and cleavage process are then analyzed to obtain information about the polynucleotide.

The information obtained from the MALDI-MS analysis typically is used to determine characteristics of the cleavable feature, e.g. identity of polynucleotides from the cleavable feature, relative concentration of the polynucleotides from the cleavable feature, presence and/or relative abundance of synthesis errors (e.g. deletions, chain terminations, depurinations, contaminating materials, or the like) or other information about the cleavable feature which may be used in evaluation of the manufacturing process. The evaluation of the manufacturing process based on the information obtained from the MALDI-MS analysis is then used to provide feedback to the manufacturing process, e.g. providing for adjustment of one or more manufacturing parameters. Such manufacturing parameters will depend on the manufacturing process, but may include one or more parameters such as concentrations of reagents, temperatures, adjustment of rinsing conditions, time (i.e. duration) of reaction, or any other such parameters as will be apparent to one of skill in the art given the disclosure herein.

Obtaining the polynucleotide bound to the substrate may be accomplished in any manner that provides the polynucleotide bound to the substrate via a cleavable linker. In typical embodiments, the cleavable linker is a triaryl methyl linker group. In an embodiment, the polynucleotide is synthesized on the substrate using previously reported synthesis methods, e.g. those reported in U.S. Pat. No. 6,222,030 to Dellinger et al., U.S. patent application Ser. No. 09/916,369 to Dellinger et al. (filed on Jul. 27, 2001), U.S. patent application Ser. No. 10/652,063 to Dellinger et al. (filed on Aug. 30, 2003. The synthesis of the polynucleotide may involve providing a functionalized substrate having a nucleotide monomer bound to the substrate via a cleavable linker, e.g. a triaryl methyl linker group, and then synthesizing a polynucleotide using the nucleotide monomer bound to the substrate as a starting point for synthesis. Given the disclosure herein, one of ordinary skill will be able to obtain the functionalized substrate and synthesize the polynucleotide on the substrate to obtain the polynucleotide bound to the substrate via a cleavable linker, e.g. a triaryl methyl linker group.

In another embodiment, the polynucleotide is procured as a polynucleotide that is in solution (not immobilized on a substrate) and is contacted with a functionalized substrate to result in the polynucleotide bound to the substrate via a cleavable linker. In such an embodiment, the functionalized substrate may have the cleavable linker bound to the substrate and a reactive group bound to the substrate via the cleavable linker. The reactive group is capable of reacting with a corresponding active group on the polynucleotide in solution, thereby immobilizing the polynucleotide on the substrate. Given the disclosure herein, one of ordinary skill will be able to obtain a functionalized substrate having a cleavable linker bound thereto and a reactive group bound to the functionalized substrate via the cleavable linker. In yet another embodiment, the polynucleotide is procured as a polynucleotide that is in solution (not immobilized on a substrate). In such an embodiment, the polynucleotide is functionalized to have a cleavable linker bound to the polynucleotide and a reactive group bound to the polynucleotide via the cleavable linker. In such an embodiment, the reactive group is capable of reacting with a corresponding active group on the substrate, thereby immobilizing the polynucleotide on the substrate. Given the disclosure herein, one of ordinary skill will be able to obtain a functionalized polynucleotide having a cleavable linker bound thereto and a reactive group bound to the functionalized polynucleotide via the triaryl methyl linker group. Any suitable reactive group capable of reacting with a corresponding active group may be used; various such groups are known in the art and may be employed by one skilled in the art given the disclosure herein.

In an embodiment, the polynucleotide is bonded to the substrate via a cleavable linker having a triaryl methyl linker group. The cleavable linker is covalently bound to the polynucleotide, e.g. directly bound or bound via an intermediate linking group. The cleavable linker is also covalently bound to the substrate, e.g. directly bound or bound via an intermediate linking group, such that the polynucleotide is bound to the substrate via the cleavable linker and via any optional intermediate linking groups. The exact structure of such intermediate linking groups is not essential to the invention, but, if present, they should provide a stable connection between the linker moiety and the substrate and/or polynucleotide. In this context, a stable connection is one that is not subject to cleavage under the conditions typically encountered during the practice of the invention. An intermediate linking group may be bonded to the adjacent cleavable linker at any position of the intermediate linking group available to bind to the adjacent cleavable linker. Similarly, an intermediate linking group may be bonded to the adjacent substrate at any position of the intermediate linking group available to bind to the adjacent substrate. Also, an intermediate linking group may be bonded to the adjacent polynucleotide at any position of the intermediate linking group available to bind to the adjacent polynucleotide. In typical embodiments, the intermediate linking groups are selected from alkyl and modified alkyl groups and combinations thereof. In certain embodiments, the intermediate linking group is a single non-carbon atom, e.g. —O— or a single non-carbon atom with one or more hydrogens attached, e.g. —N(H)—. In an embodiment, the intermediate linking group is selected from optionally substituted lower alkyl. In another embodiment, the intermediate linking group is selected from optionally substituted ethoxy, propoxy, or butoxy groups.

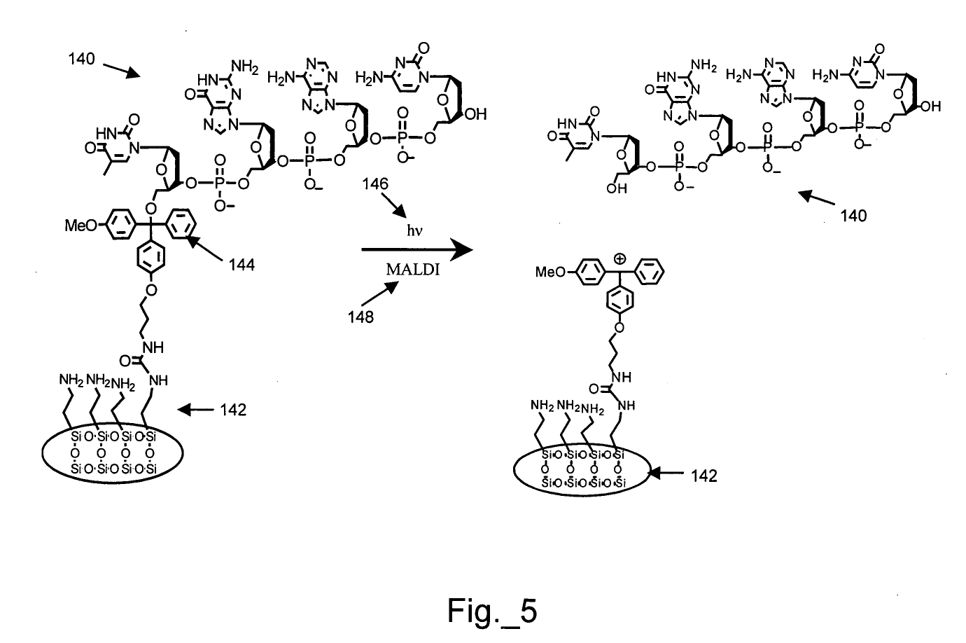

The cleavable linker is characterized as being cleavable under the conditions of the MALDI-MS analysis to release the polynucleotide from the substrate. In particular embodiments, laser radiation directed at the matrix material (which is contacting the polynucleotide) results in cleavage of the cleavable linker to release the polynucleotide from the substrate. Without being bound to any particular mechanism or limiting the invention in any way, it is believed that upon excitation of the matrix by the laser radiation, the cleavable linker, e.g. the triaryl methyl linker group, undergoes an acidic cleavage reaction to result in the polynucleotide being released from the substrate. In typical embodiments in which the cleavable linker is a triaryl methyl linker group, an acidic cleavage reaction at the central methyl carbon of the triaryl methyl group results in a triaryl methyl cation and also results in the polynucleotide being released from the substrate. At least some of the released polynucleotide will become ionized, providing ions that are analyzed by mass spectrometry to yield information about the polynucleotide. A typical reaction is shown in FIG. 5, in which a polynucleotide 140 bound to a substrate surface 142 via a triaryl methyl linker group 144 is subjected to laser radiation (“hv”) 146 during a matrix assisted laser desorption/ionization (“MALDI”) 148 process. The result of the reaction is that the polynucleotide 140 is released from the substrate surface 142. Ions derived from the polynucleotide that are desorbed and volatized are then analyzed in a mass spectrometer to yield information about the polynucleotide.

The polynucleotide, which is bonded to the substrate via the cleavable linker, typically has at least 2, at least 5, or at least 10, and may have up to 20, up to about 100, up to about 200, or even more nucleotide subunits. In certain embodiments, the polynucleotide has 2, 3, 4, or 5 nucleotide subunits. In some embodiments, the polynucleotide may have appropriate protecting groups as are known in the art of polynucleotide synthesis to prevent or reduce undesired chemical reactivity. The polynucleotide typically includes naturally occurring and/or non-naturally occurring heterocyclic bases and may include heterocyclic bases which have been modified, e.g. by inclusion of protecting groups or any other modifications described herein, or the like. The polynucleotide is typically bound to the cleavable linker via a terminal 3-O- or a 5-O- of the polynucleotide, although any other suitable site is contemplated and is within the scope of the invention.

Referring now to structure (I), the Cgp group is selected from (1) a linking group linking the substrate to the triaryl methyl linker group; or (2) a covalent bond between the substrate and the triaryl methyl linker group. In some embodiments in which Cgp is a linking group, Cgp is typically bound to a ring atom of one of the aryl groups of the triaryl methyl linker group, i.e. the Cgp group may be considered a substituent of one of the aryl groups of the triaryl methyl linker group. In other embodiments in which Cgp is a linking group, Cgp may be bound to the central methyl carbon of the triaryl methyl linker group. In some embodiments in which Cgp is a covalent bond, the substrate is typically bound to a ring atom of one of the aryl groups of the triaryl methyl linker group, i.e. the substrate may be considered a substituent of one of the aryl groups of the triaryl methyl linker group. In other embodiments in which Cgp is a covalent bond, Cgp may be bound to the central methyl carbon of the triaryl methyl linker group. In particular embodiments, the Cgp group may be any appropriate linking group (referenced herein as the Cgp linker group) that links the substrate and the triaryl methyl linker group, the Cgp linker group typically selected from (1) a lower alkyl group; (2) a modified lower alkyl group in which one or more linkages selected from ether-, oxo-, thio-, amino-, and phospho- is present; (3) a modified lower alkyl substituted with one or more groups including lower alkyl; aryl, aralkyl, alkoxyl, thioalkyl, hydroxyl, amino, sulfonyl, halo; or (4) a modified lower alkyl substituted with one or more groups including lower alkyl; alkoxyl, thioalkyl, hydroxyl, amino, sulfonyl, halo, and in which one or more linkages selected from ether-, oxo-, thio-, amino-, and phospho- is present. The Cgp linker group may be bonded to the adjacent triaryl methyl linker group at any position of the Cgp linker group available to bind to the adjacent triaryl methyl linker group. Similarly, the Cgp linker group may be bonded to the substrate at any position of the Cgp linker group available to bind to the substrate. In certain embodiments, the Cgp linker group is a single non-carbon atom, e.g. —O—, or a single non-carbon atom with one or more hydrogens attached, e.g. —N(H)—. In an embodiment, the Cgp linker group is selected from optionally substituted lower alkyl. In another embodiment, the Cgp linker group is selected from optionally substituted ethoxy, propoxy, or butoxy groups. The exact structure of the Cgp linker group is not essential to the invention, but, if present, the Cgp linker group should provide a stable connection between the triaryl methyl linker group and the substrate.

Again referring to structure (I), the Cgp′ group is selected from (1) a linking group linking the triaryl methyl linker group to the polynucleotide (typically at the terminal 5′-O or 3′-O of the polynucleotide, or other suitable site of the polynucleotide); or (2) a covalent bond between the triaryl methyl linker group and the polynucleotide (e.g. at the terminal 5′-O or 3′-O of the polynucleotide, or other suitable site of the polynucleotide). In some embodiments in which Cgp′ is a linking group, Cgp′ is typically bound to the central methyl carbon of the triaryl methyl linker group. In other embodiments in which Cgp′ is a linking group, Cgp′ may be bound to a ring atom of one of the aryl groups of the triaryl methyl linker group, i.e. the Cgp′ group may be considered a substituent of one of the aryl groups of the triaryl methyl linker group. In some embodiments in which Cgp′ is a covalent bond, the polynucleotide is typically bound to the central methyl carbon of the triaryl methyl linker group. In other embodiments in which Cgp′ is a covalent bond, Cgp′ may be bound to a ring atom of one of the aryl groups of the triaryl methyl linker group, i.e. the polynucleotide may be considered a substituent of one of the aryl groups of the triaryl methyl linker group. In particular embodiments, the Cgp′ group may be any appropriate linking group (referenced herein as the Cgp′ linker group) that links the triaryl methyl linker group to the polynucleotide, the Cgp′ linker group typically selected from (1) a lower alkyl group; (2) a modified lower alkyl group in which one or more linkages selected from ether-, oxo-, thio-, amino-, and phospho- is present; (3) a modified lower alkyl substituted with one or more groups including lower alkyl; aryl, aralkyl, alkoxyl, thioalkyl, hydroxyl, amino, sulfonyl, halo; or (4) a modified lower alkyl substituted with one or more groups including lower alkyl; alkoxyl, thioalkyl, hydroxyl, amino, sulfonyl, halo, and in which one or more linkages selected from ether-, oxo-, thio-, amino-, and phospho- is present. The Cgp′ linker group may be bonded to the adjacent triaryl methyl linker group at any position of the Cgp′ linker group available to bind to the adjacent triaryl methyl linker group. Similarly, the Cgp′ linker group may be bonded to the adjacent polynucleotide at any position of the Cgp′ linker group available to bind to the adjacent polynucleotide. In certain embodiments, the Cgp′ linker group is a single non-carbon atom, e.g. —O—, or a single non-carbon atom with one or more hydrogens attached, e.g. —N(H)—. In an embodiment, the Cgp′ linker group is selected from optionally substituted lower alkyl. In another embodiment, the Cgp′ linker group is selected from optionally substituted ethoxy, propoxy, or butoxy groups. The exact structure of the Cgp′ group is not essential to the invention, but, if present, it should provide a stable connection between the triaryl methyl linker group and the polynucleotide.

The triaryl methyl linker group in the embodiments described herein typically has the structure (II) 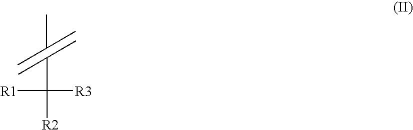
wherein the broken line represents a bond via which the triaryl methyl linker group is connected to the polynucleotide (e.g. directly or via an intermediate linking group). R1, R2, and R3 are independently selected from aromatic ring moieties (aryl groups), provided that one of R1, R2, and R3 is substituted by being bonded (e.g. directly or via an intermediate linking group) to the substrate. In other words, in a typical embodiment the substrate is bound to the central methyl carbon of the triaryl methyl linker group via one of R1, R2, and R3. Each aromatic ring moiety (aryl group) typically comprises one or more 4-, 5-, or 6-membered rings. Each aromatic ring moiety can independently be heterocyclic, non-heterocyclic, polycyclic or part of a fused ring system. Each aromatic ring moiety can be unsubstituted or substituted, e.g. substituted with one or more groups each independently selected from the group consisting of lower alkyl, aryl, aralkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, and boronyl; and lower alkyl substituted with one or more groups selected from lower alkyl, alkoxy, thioalkyl, hydroxyl thio, mercapto, amino, imino, halo, cyano, nitro, nitroso, azido, carboxy, sulfide, sulfone, sulfoxy, phosphoryl, silyl, silyloxy, and boronyl; provided that one of R1, R2, and R3 is substituted by being bound to the substrate (e.g. directly or via an intermediate linking group). In an alternate embodiment, the broken line in structure (II) represents a bond via which the triaryl methyl linker group is connected to the substrate (e.g. directly or via an intermediate linking group), and one of R1, R2, and R3 is substituted by being bound to the polynucleotide (e.g. directly or via an intermediate linking group); in such embodiments, cleavage of the linker results in the release of the triaryl methyl group from the substrate. In other words, in such embodiments the polynucleotide is bound to the central methyl carbon of the triaryl methyl linker group via one of R1, R2, or R3.

Typical triaryl methyl groups that may be employed in embodiments herein are described in U.S. Pat. No. 4,668,777 to Caruthers, again provided that, as noted above, one of R1, R2, and R3 is substituted by being bound to the substrate (or, in alternate embodiments, bound to the polynucleotide); use of such triaryl methyl groups in accordance with the present invention is within ordinary skill in the art given the disclosure herein. A substituted triaryl methyl group may have one substituent (i.e. a singly substituted triaryl methyl group) on one of the aromatic rings of the triaryl methyl group, or may have multiple substituents (i.e. a multiply substituted triaryl methyl group) on one or more of the aromatic rings of the triaryl methyl group. As used herein, an aromatic ring moiety may be referenced as an “aromatic ring structure”. As used herein, the “central methyl carbon” of a triaryl methyl group is the carbon bonded directly to the three aromatic ring structures.

In certain embodiments, R2 and R3 are each independently selected from substituted or unsubstituted aromatic groups such as phenyl, biphenyl, naphthanyl, indolyl, pyridinyl, pyrrolyl, thiophenyl, furanyl, annulenyl, quinolinyl, anthracenyl, and the like, and R1 is selected from substituted aromatic groups such as phenyl, biphenyl, naphthanyl, indolyl, pyridinyl, pyrrolyl, thiophenyl, furanyl, annulenyl, quinolinyl, anthracenyl, and the like. In some embodiments, at least one of R1, R2 and R3 is selected from substituted or unsubstituted aromatic groups other than phenyl such as naphthanyl, indolyl, pyridinyl, pyrrolyl, furanyl, annulenyl, quinolinyl, anthracenyl, and the like; in such embodiments zero, one, or two of R1, R2, and R3 are selected from substituted or unsubstituted phenyl, provided that, as noted above, one of R1, R2, and R3 is substituted by being bound to the substrate (e.g. directly or via an intermediate linking group), or, in alternate embodiments, by being bound to the polynucleotide.

In some embodiments, R1, R2, and R3 are independently selected from structure (III).

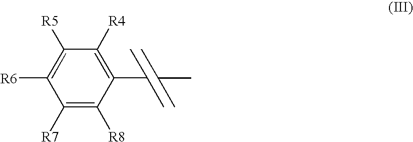

In structure (III), the broken line represents the bond to the central methyl carbon of the triaryl methyl linker group, and R4, R5, R6, R7, and R8 are each independently selected from hydrido, lower alkyl, aryl, aralkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, and boronyl; and lower alkyl substituted with one or more groups selected from lower alkyl, alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, and boronyl; provided that, for R3, one of R4, R5, R6, R7, and R8 denotes the linkage via which the triaryl methyl group is connected to one of the substrate or the polynucleotide (and the other of the substrate or polynucleotide is connected to the triaryl methyl linker group via the bond to the central methyl carbon denoted by the broken line in structure (II)).

In particular embodiments, R1, R2, and R3 are each independently selected from phenyl, methoxyphenyl, dimethoxyphenyl, and trimethoxyphenyl groups, such that the triaryl methyl linker group may be a trityl group, a monomethoxytrityl group, a dimethoxytrityl group, a trimethoxytrityl group, a tetramethoxytrityl group, a pentamethoxytrityl group, a hexamethoxytrityl group and so on; again provided as described above that one of R1, R2, and R3 is substituted by being bound (e.g. directly or indirectly) to one of the substrate or the polynucleotide (and the other of the substrate or polynucleotide is connected to the triaryl methyl linker group via the bond to the central methyl carbon denoted by the broken line in structure (II)).

In particular embodiments, R1, R2, and R3 are each independently selected from phenyl, methoxyphenyl groups, dimethoxyphenyl groups, trimethoxyphenyl groups, tetramethoxyphenyl groups, pentamethoxyphenyl groups, or furanyl groups such that the triaryl methyl linking group may be a substituted trityl group, a monomethoxytrityl group, a dimethoxytrityl group, a trimethoxyl trityl group, a tetramethoxy trityl group, a pentamethoxytrityl group, an anisylphenylfuranylmethyl group, a dianisylfuranylmethyl group, a phenyldifuranylmethyl group, an anisyldifuranylmethyl group or a trifuranylmethyl group, again provided as described above that one of R1, R2, and R3 is substituted by being bound (e.g. directly or indirectly) to one of the substrate or the polynucleotide (and the other of the substrate or polynucleotide is connected to the triaryl methyl linker group via the bond to the central methyl carbon denoted by the broken line in structure (II)).

The substrate typically comprises any material suitable for use in analysis of the polynucleotide using MALDI-MS. The material should be relatively (compared to the matrix and the polynucleotide) inert to the conditions used during the MALDI-MS analysis, e.g. exposure to laser radiation, temperature, reduced pressure, electric fields, matrix materials, etc. Typical materials include at least one material selected from the group including, but not limited to, cross-linked polymeric materials (e.g. divinylbenzene styrene-based polymers, various plastics), silica, glass, ceramics, metals, and the like, and combinations thereof.

The substrate typically has a plurality of discrete, addressable regions, each region for binding to a different polynucleotide. Typically, a region having a polynucleotide bound to the substrate via a cleavable linker is present and is available to be subjected to ionization and analysis by MALDI-MS in accordance with the method of the present invention. Typically, the number of addressable regions present on the substrate ranges from about 1 to about 400, up to about 1600 or more, for example as many as about 3000, 5000, 10,000 or more discrete addressable regions may be present on a single substrate. The substrate may also have elements that serve to confine or locate the polynucleotide and/or other substances (e.g. matrix materials or other reagents) on the substrate. Such elements may include wells or depressions on the surface of the substrate, or a hydrophobic (or hydrophilic) pattern on the surface, or a visible grid pattern. The configuration or pattern of such elements may vary depending on the particular MALDI protocol being employed, the number of features present, the size and shape of the features present, etc. The configuration of the features of the substrate may be in a grid format or other analogous geometric or linear format or the like, e.g., similar to a conventional microtiter plate grid pattern; in certain embodiments the features are present in a non grid-like or non-geometric pattern.

In general, the substrate may be any shape, and the choice of shape is generally defined by the shapes acceptable to the mass spectrometer employed in the subject methods. In particular embodiments, the substrate may have a square, rectangular, or circular shape, with one or more discrete addressable regions (e.g. features) arranged in a parallel, random, spiral, grid configuration or any other configuration that can be accommodated on a surface of the substrate.

Typically the substrate has a surface to which the polynucleotide is bound via the cleavable linker. In certain embodiments, the substrate comprises a solid support and a modification layer disposed on (or bound to, e.g. directly or indirectly) the solid support, and the cleavable linker is bound to (e.g. directly or indirectly) the modification layer. Such modification layer may be formed on the substrate by methods known in the art to modify the surface properties of the solid support. The solid support typically comprises the same or similar materials or combinations of materials used to describe the substrate herein. In certain embodiments, the modification layer may be, e.g. a coating, a material deposited by deposition techniques known in the art, a hydrophobic layer, or a hydrophilic layer. In particular embodiments, the modification layer comprises a silane group, to which the cleavable linker is bound, directly or indirectly, e.g. via any linking group effective to link the cleavable linker to the silane group and stable to the conditions used in the methods described herein. Particularly contemplated are modification layers taught in U.S. Pat. No. 6,258,454 to Lefkowitz et al. (2001), which describes a moiety bound to a substrate via a linking group attached to a silane group bound to the substrate.

Substrates in accordance with the present invention may be made using silane modified substrates such as are employed in the Lefkowitz '454 patent and modifications thereof. In such methods, an available reactive group attached (directly or indirectly, e.g. via a linking group) to the silane group on the substrate provides a site for further attachment to the substrate to occur. Methods of preparing substrates having triaryl methyl linker groups bound to the solid support are taught in U.S. patent application Ser. No. 10/652,063 to Dellinger et al., filed on Aug. 30, 2003. The resulting functionalized substrate may be used for in situ synthesis of a polynucleotide or to bind to a pre-synthesized polynucleotide, as explained herein. Selection and preparation of the substrate will be based on experimental design considerations, such as the desired available reactive group attached to the substrate, number of different polynucleotides to be analyzed, design considerations for facilitating deposition of reagents such as polynucleotide, matrix materials, or other reagents, etc. Such selection and preparation is within the skill of those in the art given the disclosure herein.

The substrate may also have features that serve to aid in the MS analysis, e.g. electrically conductive materials coating the surface of the substrate or forming a conductive pattern (such as a grid) on the substrate. In typical embodiments, the substrate is a MALDI sample plate. In general, MALDI sample plates with a plurality of fluid retaining structures are known and described in U.S. Patent Publication Serial Nos. 20030057368, and 20030116707. For example, e.g., “anchor” sample plates that have hydrophobic and/or hydrophilic coatings (see, e.g., U.S. Pat. No. 6,287,872) are well known and purchasable in 96 sample and 384 sample formats from Bruker Daltonik (Germany). Other suitable MALDI sample plates are purchasable from Agilent Technologies (Palo Alto, Calif.).

In certain embodiments, an array including many features on a substrate, wherein at least one feature is a cleavable feature having a polynucleotide bound to the substrate via a triaryl methyl linker group, may be provided by a method comprising using a fluid delivery device to deliver reagents, analytes (e.g. polynucleotides), matrix materials, etc. to the substrate surface. The fluid delivery device may be, e.g. a pulse-jet fluid delivery device or a contact fluid delivery device. The fluid delivery device, in certain embodiments, may also be employed to perform in situ synthesis of the polynucleotides on the substrate surface. Suitable fluid delivery devices include pulse-jet printing devices, and contact printing devices such as pipetting robots, capillary printers, and the like. Suitable pipetting robots may be used to perform all of the steps described above. Typical examples of pipetting robots include the following systems: GENESIS™ or FREEDOM™ of Tecan (Switzerland), MICROLAB 4000™ of Hamilton (Reno, Nev.), QIAGEN 8000™ of Qiagen (Valencia, Calif.), the BIOMEK 2000™ of Beckman Coulter (Fullerton, Calif.) and the HYDRA™ of Robbins Scientific (Hudson, N.H.). In particular embodiments, pulse-jet printing devices such as piezoelectric devices may be used (see e.g., Li et al., J. Proteome Res. (2002) 1:537-547; Sloan et al., Molecular and Cellular Proteomics (2002) 490-499).

The array provided in accordance with the invention is capable of being placed in operable association in a MALDI source apparatus, e.g. the array is configured as a MALDI sample plate and may be inserted into the MALDI source of a mass spectrometer. The array is then subjected to analysis by MALDI-MS to assess the polynucleotide bound to the substrate surface via a triaryl methyl linker group. Accordingly, the invention provides a method for assessing a cleavable feature of an array. In certain embodiments, the same polynucleotide may be present in two or more different regions of the substrate (e.g. different features of the array). Typically, a plurality of different polynucleotides are bound to the substrate, each polynucleotide bound at its own addressable region. In this case, the resulting substrate (e.g. MALDI sample plate) will usually contain a plurality of regions containing different polynucleotides to be analyzed. Each region is then contacted with the matrix material, which is allowed to dry to form crystals, e.g. thus forming a prepared MALDI sample plate containing analytes that is suitable for use in a MALDI mass spectrometer. In some embodiments, a plurality of polynucleotides are bound at the same addressable region of the substrate such that the plurality of polynucleotides are analyzed simultaneously in the mass spectrometer. In some embodiments, one or more of the addressable regions will not be bound to a polynucleotide.

Prior to analysis by mass spectrometry, the polynucleotide bound to the substrate is typically contacted with an energy absorbing matrix material, as is known in the art. The matrix material is typically a small organic, volatile compound with certain properties that facilitate the performance of MALDI. Accordingly, a matrix material is selected based on a variety of factors such as the analyte of interest (such as type or size of molecule), and the like. For example, a matrix material is selected that allows the cleavage of the triaryl linker and release of the polynucleotide from the substrate. Further, a matrix material should be selected that provides for generation of a sufficient quantity of ions to be analyzed in a mass spectrometer to obtain information about the polynucleotide.

Examples of matrix materials include, but are not limited to, sinapinic acid (SA) and derivatives thereof, such as alpha-cyano sinapinic acid; cinnamic acid and derivatives thereof, such as 3,5-dimethoxy-4-hydroxycinnamic acid; 2,5-dihydroxybenzoic acid (DHB); and dithranol. Further examples of matrices that are typical for use with polynucleotide analytes include 3-hydroxy-picolinic acid (HPA); 2,4,6-trihydroxyacetophenone (246THAP); 4-hydroxy-3-methoxycinnamic acid (Ferulic acid); trans-Indole-3-acrylic acid (IAA); 2,3,4-trihydroxyacetophenone (234THAP); 4-hydroxy-alpha-cyano-cinnamic acid methyl ester. In some embodiments, mixtures of two or more of the materials listed in this paragraph (or yet other matrix materials known in the art) may be used as the matrix material in the methods of the present invention. The desired matrix material (or combination of matrix materials) is typically dissolved in a suitable solvent that is selected at least in part based on suitability for applying the matrix material to the substrate to gain good contact between the matrix material and the polynucleotide and the triarylmethyl linker group. For example, in the analysis of oligonucleotides, 3-hydroxy-picolinic acid (HPA) dissolved in a solvent of acetonitrile and water may be employed. After application of the matrix material to the substrate, e.g. contacting a site on the substrate having a polynucleotide bound thereto, the matrix material is allowed to dry to form crystals.

The polynucleotide may be analyzed using any mass spectrometer that has the capability of measuring masses with a desired level of mass accuracy, precision, and resolution. Accordingly, the polynucleotides may be analyzed by any one of a number of mass spectrometry methods, including, but not limited to, matrix-assisted laser desorption ionization time-of-flight mass spectrometry (MALDI-TOF) and any tandem MS such as QTOF, TOF-TOF, etc. The mass spectrometry protocol may be an atmospheric pressure (AP) MALDI protocol or a vacuum MALDI protocol. Mass spectrometry methods are generally well known in the art (see Burlingame et al. Anal. Chem. 70:647R-716R (1998); Kinter and Sherman, Protein Sequencing and Identification Using Tandem Mass Spectrometry Wiley-Interscience, New York (2000)). Any convenient MALDI protocol may be adapted and employed with the subject invention. Representative MALDI protocols, as well as apparatuses for use in performing MALDI protocols, that may be adapted for use with the subject invention include, but are not limited to, those described in International Publication Nos.: GB 2,312782 A; GB 2,332,273 A; GB 2,370114A; and EP 0964427 A2, as well as in U.S. Patent Publication No. 2002031773; and U.S. Pat. Nos. 5,498,545; 5,643,800; 5,777,324; 5,777,860; 5,828,063; 5,841,136; 6,111,251; 6,287,872; 6,414,306; and 6,423,966 The basic processes associated with a mass spectrometry method are the generation of gas-phase ions derived from the sample, and the measurement of their mass. The analysis by MALDI-MS typically provides information about the polynucleotides, such as the mass of the isolated analytes or fragments thereof, and their relative or absolute abundances in the sample, information about identity of the polynucleotide, etc.

The analysis by MALDI-MS typically includes evaluation of the data obtained from mass spectrometry analysis. For example, molecular mass data may be compared against expected values for known or anticipated analytes. The evaluation of the molecular mass data may involve the elimination of signals obtained that are not derived from the analytes of interest, so that only those signals corresponding to the pre-determined analytes may be retained. In many embodiments, the masses of the analytes or fragments thereof are stored in a table of a database and the table usually contains at least two fields, one field containing molecular mass information, and the other field containing analyte identifiers, such as names or codes. As such, the subject methods may involve comparing data obtained from mass spectrometry to a database to identify data for an analyte of interest. In general, methods of comparing data produced by mass spectrometry to databases of molecular mass information to facilitate data analysis is very well known in the art (see, e.g., Yates et al., Anal. Biochem. (1993) 214:397-408; Mann et al., Biol Mass Spectrum. (1993) 22:338-45; Jensen et al., Anal. Chem. (1997) D69:4741-50; and Cottrell et al., Pept Res. 1994 7:115-24) and, as such, need not be described here in any further detail. Accordingly, information, e.g., data, regarding the amount of analytes in a sample of interest (including information on their presence or absence) may be obtained using mass spectrometry.

As is well known in the art, for each analyte, information obtained using mass spectrometry may be qualitative (e.g., showing the presence or absence of an analyte, or whether the analyte is present at a greater or lower amount than a control analyte or other standard) or quantitative (e.g., providing a numeral or fraction that may be absolute or relative to a control analyte or other standard). Also as is known, standards for assessing mass spectrometry data may be obtained from a control analyte that is present in the isolated analytes, such as an analyte of known concentration, or an analyte that has been added at a known amount to the isolated analytes, e.g., a spiked analyte. Accordingly, the data produced by the subject methods may be “normalized” to an internal control, e.g. an analyte of known concentration or the like.

By comparing the results from assessing the presence of an analyte in two or more different samples using the methods set forth above, the relative levels of an analyte in two or more different samples may be obtained. In other embodiments, by assessing the presence of at least two different analytes in a single sample, the relative levels of the analytes in the sample may be obtained.

In typical embodiments, a polynucleotide is analyzed by mass spectrometry, and, by integrating the signals produced by the ions derived from the polynucleotide, measurements corresponding to the abundance of particular ions are provided. Using software that is already available and commonly used to identify ion masses, the data is usually compared to a database of ion masses expected for the polynucleotides. By doing this comparison, the identity and abundance of the polypeptide corresponding to a particular ion becomes known. Depending on the exact method used, a table containing data on the abundance of ions (or the corresponding polynucleotides) may be exported to a separate database, and saved.

The information obtained from the MALDI-MS analysis typically is used to determine characteristics of the cleavable feature, e.g. identity of polynucleotides from the cleavable feature, relative concentration of the polynucleotides from the cleavable feature, presence and/or relative abundance of synthesis errors (e.g. deletions, chain terminations, depurinations, contaminating materials, or the like) or other information about the cleavable feature which may be used in evaluation of the array manufacturing process. The evaluation of the manufacturing process based on the information obtained from the MALDI-MS analysis is then used to provide feedback to the manufacturing process, e.g. providing for adjustment of one or more manufacturing parameters. Such manufacturing parameters will depend on the manufacturing process, but may include one or more parameters such as concentrations of reagents, temperatures, adjustment of rinsing conditions, time (i.e. duration) of reaction, or any other such parameters as will be apparent to one of skill in the art given the disclosure herein.

### EXAMPLES

The practice of the present invention will employ, unless otherwise indicated, conventional techniques of synthetic organic chemistry, biochemistry, molecular biology, and the like, which are within the skill of the art. Such techniques are explained fully in the literature.

The following examples are put forth so as to provide those of ordinary skill in the art with a complete disclosure and description of how to perform the methods and use the compositions disclosed and claimed herein. Efforts have been made to ensure accuracy with respect to numbers (e.g., amounts, temperature, etc.) but some errors and deviations should be accounted for. Unless indicated otherwise, parts are parts by weight, percents are wt./wt., temperature is in ° C. and pressure is at or near atmospheric. Standard temperature and pressure are defined as 20° C. and 1 atmosphere.

Abbreviations used in the examples include: THF is tetrahydrofuran; TLC is thin layer chromatography; HEX is hexane; Et₃N is triethylamine; MW is molecular weight; AcCN is acetonitrile; sat'd is saturated; EtOH is ethanol; B is a heterocyclic base having an exocyclic amine group, B^{Prot} is a heterocyclic base having an exocyclic amine group with a trityl protecting group on the exocyclic amine group; TiPSCl is 1,3-dichloro-1,1,3,3-tetraisopropyldisiloxane; TEMED is N,N,N′,N′-Tetramethylethylenediamine; Py is pyridine; MeCN is acetonitrile; DMT is dimethoxytrityl; MMT is monomethoxytrityl; TMT is trimethoxytrityl; Cyt^{DMT} is cytosine which has a dimethoxytrityl protecting group on the exocyclic amine group; Cyt^{TMT} is cytosine which has a trimethoxytrityl protecting group on the exocyclic amine group (and so on for other bases and protecting groups on the exocyclic amine group of the indicated base); MS is mass spectrometry, MS (ES) is mass spectrometry (electrospray), HRMS (FAB) is high resolution mass spectrometry (fast atom bombardment); DCM is methylene chloride; EtOAc is ethyl acetate; ⁱPr is isopropyl; Et₃N is triethylamine; TCA is trichloroacetic acid; TEAB is tetraethylammonium bicarbonate.

A synthesis of reagents used in certain embodiments of the present invention is now described. It will be readily apparent that the reactions described herein may be altered, e.g. by using modified starting materials to provide correspondingly modified products, and that such alteration is within ordinary skill in the art. Given the disclosure herein, one of ordinary skill will be able to practice variations that are encompassed by the description herein without undue experimentation.

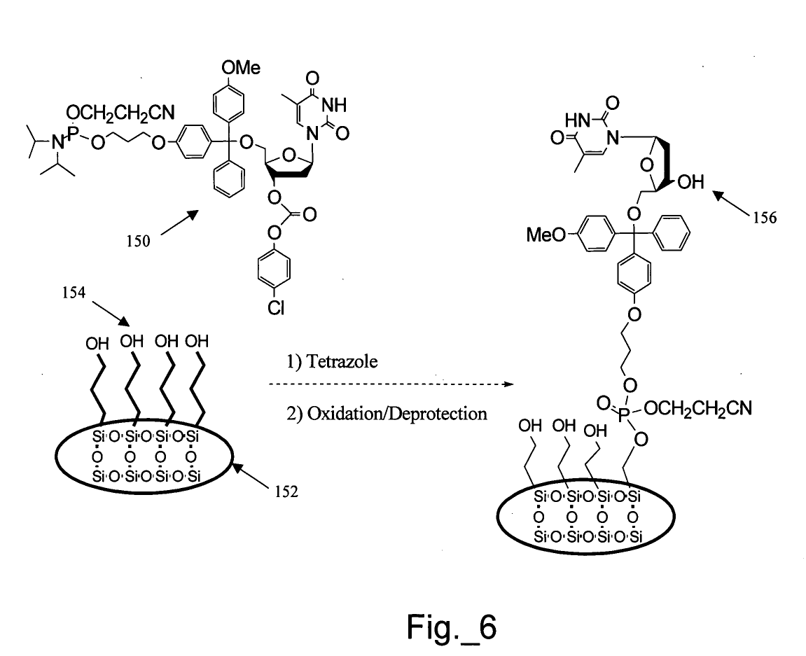

The triaryl methyl linker can be synthesized as a phosphoramidite (e.g. step 6, below) and reacted with a hydroxyl containing surface of a substrate (e.g. by inkjet deposition of the linker phosphoramidite onto the surface of the substrate) to produce the cleavable linker bound to the substrate at any number of sites on the substrate (see FIG. 6).

### 4-Hydroxy-4′-Methoxytrityl Alcohol

### Step 1

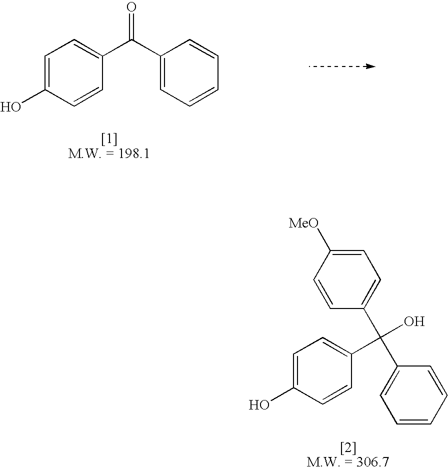 [0116] (A) 25.0 g (126.2 mmoles) 4-hydroxy Benzophenone (1); Aldrich # H2020-2
(B) 500 ml THF; Aldrich # 49446-1
(C) 700 ml of a 0.5 M Solution in THF (175 mmoles) 4-Anisyl Magnesium Bromide; Alpha-Aesar # 89435
   TLC System: HEX/EtOAc/Acetone (4:1:1)+0.5% Et₃N on silica gel

Using a 3-L 3-neck round bottom flask with a mechanical stirrer, U-tube thermometer and drying tube, (A) was added to (B) and the solution was cooled to 4° C. in a dry-ice/acetone bath, under Argon atmosphere. (C) was added drop wise over a period of 1 hour. Precipitate forms tan→pink color. The temperature was kept between 0-5° C. during the addition. The mixture was removed from the bath and stirred at ambient temperature (under Argon atmosphere) for 16-hours. The solvent was evaporated in vacuo. The residue was suspended in 300 ml ether and 200 mL cold water. The ether layer was extracted with 150 mL saturated NaHCO₃ and 150 mL saturated NaCl and dried with MgSO₄. The solvent was evaporated, and 66 g of an oily residue was obtained. The residue was dissolved in 50 mL DCM, 30 g silica gel added and column purified over silica gel, with DCM/AcCN (19:1) as the initial mobile phase, changing to DCM/AcCN (9:1) as mobile phase for elution of the product. The product was column purified a second time over silica gel using EtOAc/HEX (1:1) as mobile phase for elution of the product.

Theoretical Yield: 38.6 g

Actual Yield: 23.9 g [62%]

¹H NMR (CDCl₃) 3.78 (3H, s), 6.75 (2H, d, J=8.8), 6.83 (2H, d, J=8.8), 7.11 (2H, d, J=8.8), 7.17 (2H, d, J=8.8), 7.25-7.32 (5H, m); MS (ESI-) m/z 305 (M−1, 100); (ESI+) m/z 635 (M₂+Na, 33), 289 (M−H₂O, 100)

### 4-((3-Propoxy)-tert-Butyldimethylsilane)-4′-Methoxytrityl Alcohol

### Step 2

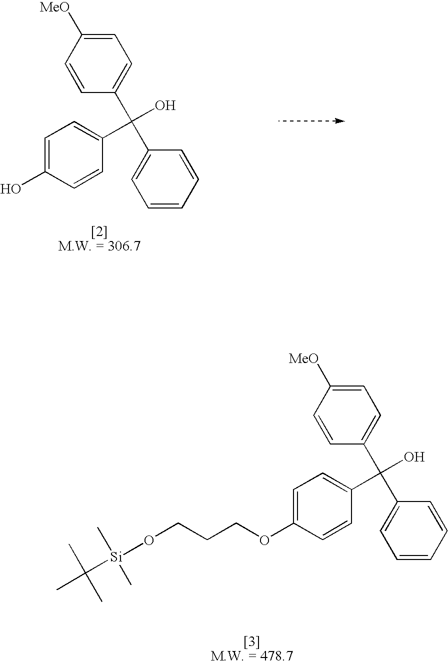 TLC System: DCM/AcCN [19:1]
(A) 24.0 g (78.0 mmoles) [2]
(B) 21.6 g (156 mmoles) potassium carbonate MW=138.1; Aldrich # 20961-9
(C) 60 g (235 mmoles) (3-Bromopropoxy)-tert-butyldimethylsilane MW=253.3; Aldrich # 42,906-6
(D) Single (Dry) Crystal Potassium Iodide MN 166.1; Aldrich # 22194-5
(E) 600 mL Toluene

Using a 2 L 3-neck round bottom flask equipped with a thermometer, reflux condenser, drying tube and stir bar, (A), (B) (C), and (D) were added to (E) in sequential order. The mixture was heated to reflux for 24 hours. The solvent was evaporated. The residue was partitioned between 750 mL DCM and 300 mL water. The DCM layer was washed twice with 400 mL sat'd NaCl then dried over MgSO₄.

Theoretical Yield: 37.3 g

Actual Yield: 16 g [43%]

MS (FAB+) m/z 479, 462 (M—OH, 100)

### 4-((3-Propoxy)-tert-Butyldimethylsilane)-4′-Methoxytrityl Chloride

### Step 3

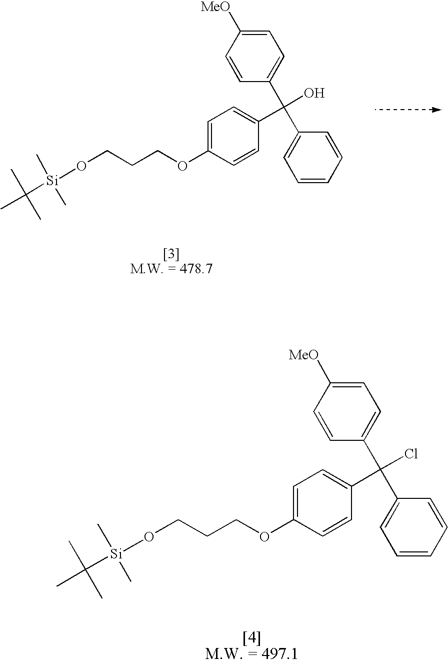 TLC System: Hexane/EtOAc [2:1]
(A) 5.0 g (10.44 mmol) [3]
(B) 18.2 mL (208 mmol) oxalyl chloride MW=126.9; Aldrich # 32042-0
(C) 150 mL Hexane

A 250 mL 3-neck round bottom flask was equipped with a cold-finger reflux/distillation condenser, magnetic stir bar, and two silicon rubber septa. (A) was suspended in (C) in the flask, and the flask was placed under argon and stirred. (B) was added to the stirring solution drop wise. Upon addition the suspended material dissolved and small bubbles formed in the flask. The reaction was refluxed overnight. The next morning the refluxing reaction consisted of a clear refluxing solution and a viscous orange-red oil on the bottom of the flask. The condenser was then set to distill and the hexanes and excess (B) removed by distillation. The remaining oil was placed under high vacuum resulting in 6.7 g of a foamed solid, used in the following reaction.

Theoretical Yield: 5.2 g

Actual Yield 5.2 g [100%]

### 3′-O-(4-Chlorophenyl)-Carbonyl-2′-Deoxythymidine

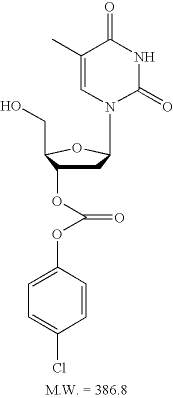

5′-O-(4,4′-Dimethoxytrityl)-2′-deoxythymidine (10.89 g, 20.0 mmol) was coevaporated from pyridine (3×40 mL), dissolved in pyridine (180 mL), and 4-chlorophenyl chloroformate (3.06 mL, 24.0 mmol) added with vigorous stirring. The mixture was stirred for 2 hours, solvent removed in vacuo, and the oily residue coevaporated with toluene (100 mL). The resulting oil was dissolved in dichloromethane (500 mL), extracted with sat. NaHCO₃ (250 mL) and brine (250 mL), dried over MgSO₄, and solvent evaporated to yield a viscous yellow oil. Purification by silica gel chromatography (0-2% ethanol in 100:0.1 dichloromethane:triethylamine) yielded 3′-O-(4-chlorophenyl)-carbonyl-5′-O-(4,4′-dimethoxytrityl)-2′-deoxythymidine as a white, glassy solid (10.93 g, 78.2%).

Anal. ¹H NMR (400 MHz, CDCl₃) δ 9.27 (1H, s, H₃), 7.63 (1H, s, H₆), 6.85-7.42 (17H, m), 6.54 (1H, m, H_{1′}), 5.43 (1H, m, H_{3′}), 4.32 (1H, m, H_{4′}), 3.78 (6H, s), 3.44-3.59 (2H, m, H_{5′}), 2.47-2.68 (2H, m, H_{5′,5″}), 1.40 (3H, s); ¹³C NMR (100.5 MHz, CDCl₃) δ 163.7, 158.8, 152.7, 149.7, 149.2, 144.1, 135.1, 135.0, 131.7, 130.1, 130.0, 129.8, 129.6, 128.0, 127.2, 122.2, 113.3, 111.7, 87.3, 84.3, 83.6, 79.9, 63.6, 55.2, 37.8, 11.6; MS (FAB+) m/z 698 (M, 100).

To 3′-O-(4-chlorophenyl)-carbonyl-5′-O-(4,4′-dimethoxytrityl)-2′-deoxythymidine (2.50 g, 3.58 mmol) was added a 3% solution of trichloroacetic acid in dichloromethane (400 mL) with vigorous stirring. The mixture was stirred for 3 min before pyridine/methanol (1:1) was added drop wise until the red color of the DMT cation was quenched. The mixture was extracted with saturated NaHCO₃ (300 mL) and brine (300 mL), dried over MgSO₄, and solvent removed in vacuo. Purification of the resulting oil by silica gel chromatography (0-6% ethanol in dichloromethane) afforded the 3′-O-(4-Chlorophenyl)-Carbonyl-2′-Deoxythymidine as a white powder (1.30 g, 92%);

Anal. Calcd. for C₁₇H₁₇ClN₂O₇: C, 51.5; H, 4.3; N, 7.1. Found: C, 51.3; H, 4.5; N, 7.0. ¹H NMR (400 MHz, CDCl₃/d₄-MeOH) 9.57 (1H, s, H₃), 7.44 (1H, s, H₆), 7.25 (2H, d, J=8.8), 7.03 (2H, d, J=8.8), 6.17 (1H, m, H_{1′}), 5.27 (1H, m, H_{3′}), 4.17 (1H, m, H_{4′}), 3.83 (2H, m, H_{5′}), 2.42 (2H, m, H_{2′,2″}), 1.80 (3H, s); ¹³C NMR (100.5 MHz, CDCl₃/d₄-MeOH) 164.1, 152.8, 150.6, 149.2, 136.7, 131.7, 129.6, 122.2, 111.4, 86.3, 84.8, 79.5, 62.4, 37.0, 12.5; MS (ESI+) m/z 397 (M+1, 100).

### 5′-O-4-((3-Propoxy)-tert-Butyldimethylsilane)-4″-Methoxytrityl-3′-O-(4-Chlorophenyl)-Carbonyl-2′-Deoxythymidine

### Step 4

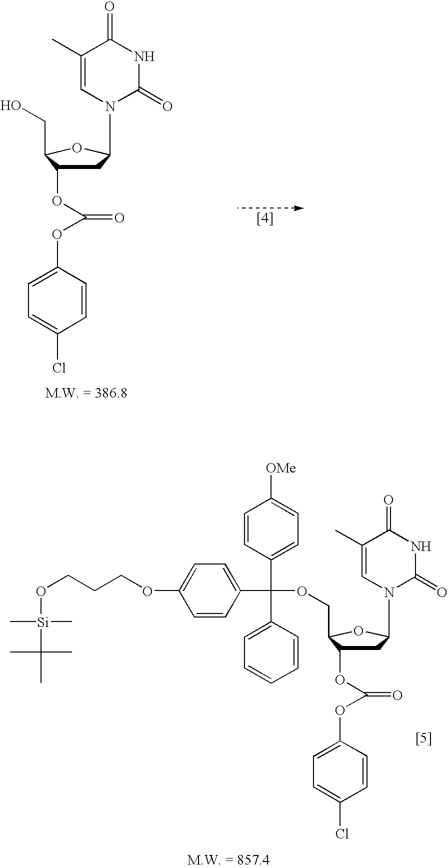

To 3′-O-(4-chlorophenyl)-carbonyl-2′-deoxythymidine (1.2 g, 3.1 mmol) in pyridine (35 mL) was added 4-((3-Propoxy)-tert-Butyldimethylsilane)-4′-Methoxytrityl Chloride (1.86 g, 3.75 mmol). The mixture was stirred for 4 h at which point the solvent was removed under reduced pressure. The residue was dissolved in dichloromethane, washed with 5% sodium carbonate and brine, dried (MgSO₄), and solvent removed in vacuo to yield a pale yellow oil. The 5′-O-4-((3-Propoxy)-tert-Butyldimethylsilane)-4″-Methoxytrityl-3′-O-(4-Chlorophenyl)-Carbonyl-2′-Deoxythymidine was isolated by silica gel chromatography using 1-4% methanol/dichloromethane as eluant as a pale yellow glassy solid (2.4 g, 90.0%); MS (FAB+) m/z 743 (M, 100).

### 5′-O-4-(3-Hydroxypropyl)-4″-Methoxytrityl-3′-O-(4-Chlorophenyl)-Carbonyl-2′-Deoxythymidine

### Step 5

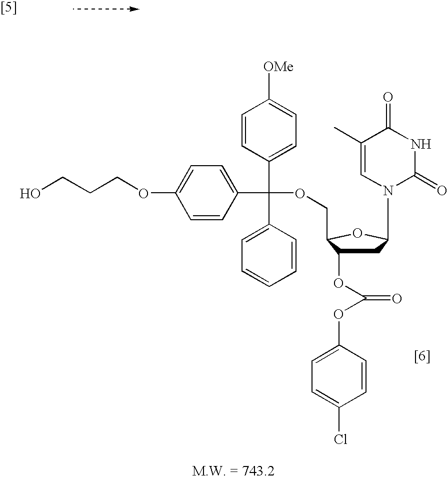

5′-O-4-((3-Propoxy)-tert-butyldimethylsilane)-4″-methoxytrityl-3′-O-(4-chlorophenyl)-carbonyl-2′-deoxythymidine (2.4 g, 2.8 mmol) was dissolved in anhydrous pyridine (75 mL) using a magnetic stirrer. The flask was kept anhydrous under argon and cooled in an ice/water bath. Hydrogen fluoride pyridine (100 μL) Fluka cat# 47586 was dissolved in 10 mL of anhydrous pyridine and added to the stirring flask. The reaction was allowed to stir for 30 min then evaporated to a rust brown oil. The residue was dissolved in dichloromethane, washed with 5% sodium carbonate and brine, dried (MgSO₄), and solvent removed in vacuo to yield a dark yellow oil. The 5′-O-4-(3-Hydroxypropyl)-4″-Methoxytrityl-3′-O-(4-Chlorophenyl)-Carbonyl-2′-Deoxythymidine was isolated by silica gel chromatography using 0-3% methanol/dichloromethane as eluant as a pale yellow glassy solid (2.4 g, 90.0%); MS (FAB+) m/z 859 (M, 100).

### 5′-O-4-(3-propyloxy(2-Cyanoethyl N,N-diisopropylphosphoramidite))-4′-Methoxytrityl-3′-O-(4-Chlorophenyl)-Carbonyl-2′-Deoxythymidine Step 6

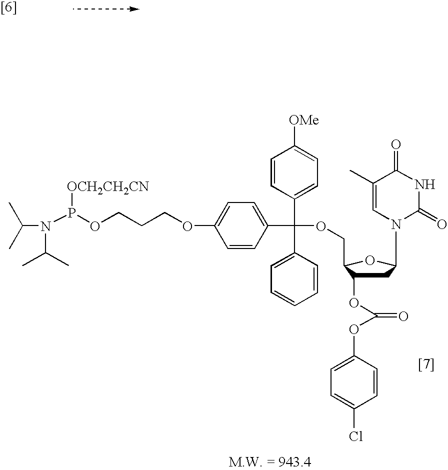

5′-O-4-(3-Hydroxypropyl)-4″-Methoxytrityl-3′-O-(4-Chlorophenyl)-Carbonyl-2′-Deoxythymidine 3.7 g (5.0 mmol) and tetrazole (175 mg, 2.50 mmol) were dried under vacuum for 24 h then dissolved in dichloromethane (100 mL). 2-Cyanoethyl N,N,N′,N′-tetraisopropylphosphorodiamidite (2.06 mL, 6.50 mmol) was added in one portion and the mixture stirred over 1 h. The reaction mixture was washed with sat. NaHCO₃ (150 mL) and brine (150 mL), dried over MgSO₄, and applied directly to the top of a silica column equilibrated with hexanes. The dichloromethane was flashed off the column with hexanes, and the product eluted as a mixture of diastereoisomers using 1:1 hexanes:ethyl acetate then ethyl acetate. After evaporation of solvents in vacuo and coevaporation with dichloromethane, product was isolated as friable, white, glassy solids in 75% yield; ³¹P NMR (162.0 MHz, CDCl₃) 148.89, 148.85; MS (FAB+) m/z 945 (FAB-) m/z 943

It will be apparent to one of skill in the art that the series of syntheses described above may be altered to employ analogous starting materials that react in a similar manner to give analogous products, and that such alteration of the synthesis is within ordinary skill in the art. For example, thymidine may be replaced with N-4-dimethoxy trityl-2′-deoxycytidine in step 4 to give 5′-O-4-(3-propyloxy-(2-cyanoethyl N,N-diisopropyl-phosphoramidite))-4″-methoxytrityl-3′-O-(4-chlorophenyl)-carbonyl-N-4-dimethoxytrityl-2′-deoxycytidine as the final product. As another example, in step 2, the (3-bromopropoxy)-tert-butyldimethylsilane may be replaced with (4-bromobutoxy)-tert-butyldimethylsilane to give 4-((4-Butoxy)-tert-butyldimethylsilane)-4′-methoxytrityl alcohol the product of step 2. As another example, it will be appreciated that the nucleoside moiety may be bound to the triaryl methyl linker group via either the 3′-OH or the 5′-OH. Such a modification will be accomplished by reacting a 5′-O-protected nucleoside with the trityl linker under conditions that enhance the rate of trityl reaction with secondary hydroxyls such as the addition of an acylation catalyst like N,N-dimethlyaminopyridine or silver salts as well as other techniques well known to one skilled in the art.

Furthermore, in the reaction designated as “Step 1”, above, the starting materials may be modified to yield a product wherein one or more of the phenyl (or substituted phenyl) rings is replaced by an alternate aromatic ring moiety, such as substituted or unsubstituted aromatic groups such as phenyl, biphenyl, naphthanyl, indolyl, pyridinyl, pyrrolyl, thiophenyl, furanyl, annulenyl, quinolinyl, anthracenyl, and the like. Such products may then be used as alternative starting materials in the reaction designated “Step 2” (and so on through the rest of the described syntheses) to give a triaryl methyl-modified nucleotide monomer, above.

As shown in the reaction illustrated in FIG. 6, the 5′-linked molecules 150 can then be reacted with a substrate 152 having a reactive moiety 154 such as a hydroxyl group, thiol group, or amino group, wherein the substrate 152 is suitable for use for polynucleotide synthesis. The 3′-hydroxyl 156 of the nucleoside moiety may then be used as a starting point for performing cycles of a polynucleotide synthesis reaction to give a product in which a polynucleotide strand is bound to the substrate via the trityl group. An example of such a product is shown in FIG. 5 in which an oligonucleotide that is four nucleotides long has been synthesized and is bound to the substrate via the trityl moiety.

The reaction illustrated in FIG. 6 (or similar reactions apparent to those of ordinary skill given the disclosure herein) may be conducted at one or more regions of an array substrate, followed by cycles of a polynucleotide synthesis reaction at each region, to provide for one or more cleavable features of the fabricated array provided in accordance with the method of the present invention. Once the synthesis is complete, the substrate and the attached polynucleotide can be contacted with a matrix material and then subjected to analysis by MALDI-MS.

### MALDI/TOF Analysis of Trityl Linker on Planar Glass Surface

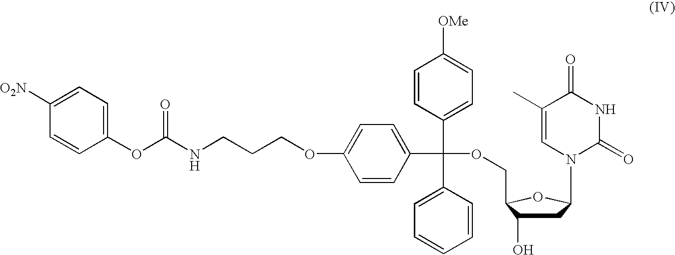

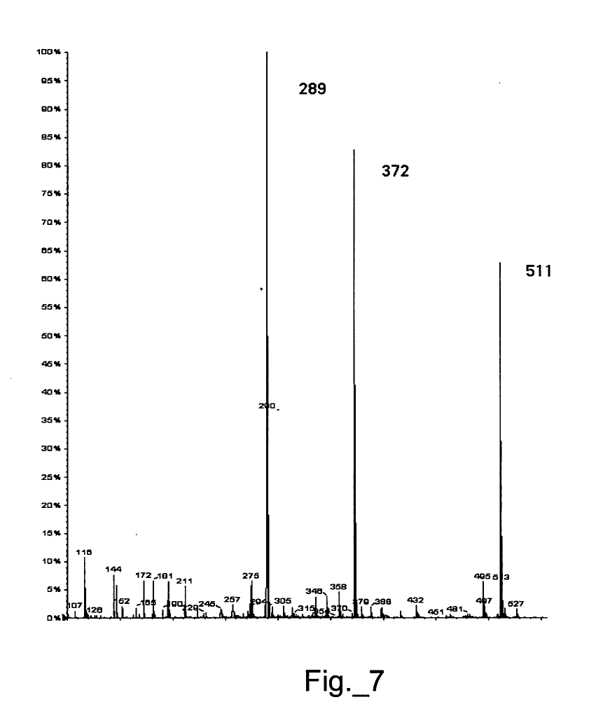

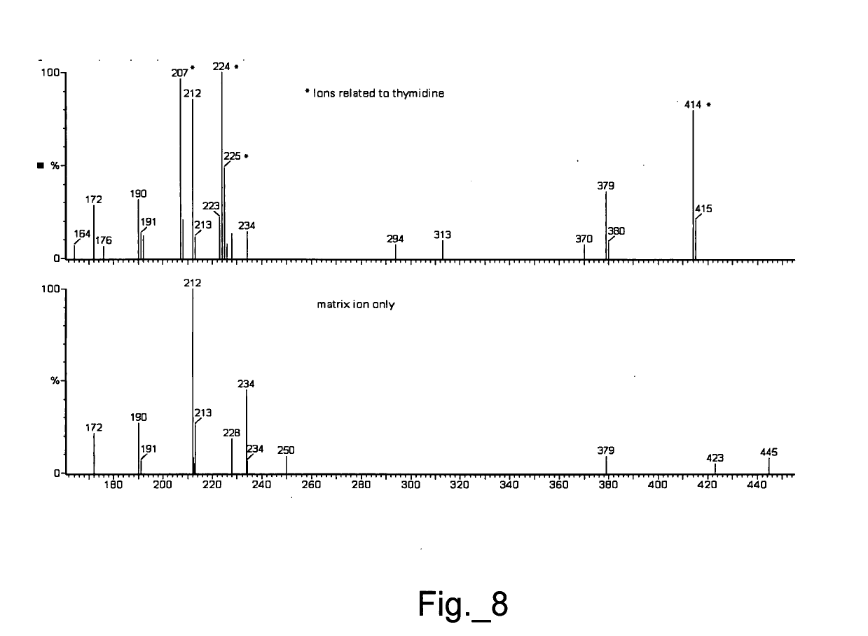

MALDI analysis of structure (IV) in positive ion mode with alpha-cyano-4-hydroxycinnamic acid, DHB or sinapinic acid as the matrix gives the mass spectra shown in FIG. 7. The prominent signals in FIG. 7 were assigned the following structures: 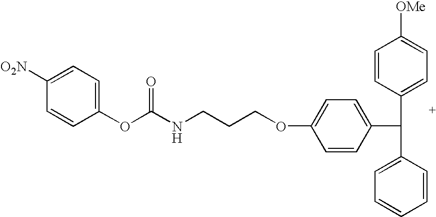
m/e 511 loss of thymidine 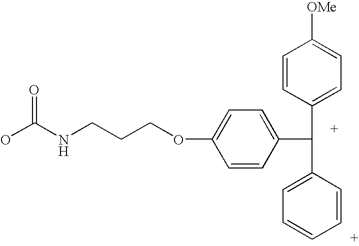
m/e 372 loss of nitrobenzene from 511

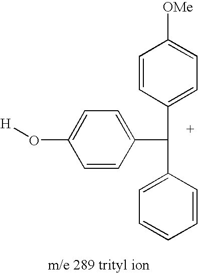

In negative ion mode no ions other than matrix ions were detected. Alpha-Cyano-4-Hydroxycinnamic Acid, DHB and sinipinic acid are not considered to be good matrices for generating negative ions. Further choices of MALDI matrices potentially capable of supporting negative ion generation include the compounds 3-HPA, 234THAP, 246THAP, and IAA.

MALDI of the ALTA linker with thymidine (see FIG. 6) in positive ion mode using Alpha-Cyano-4-Hydroxycinnamic Acid as the matrix gives the mass spectra seen in FIG. 8. The prominent signals in FIG. 8 were assigned structures as follows: 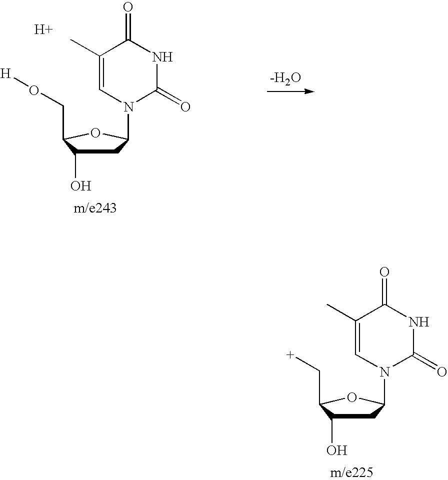
225 Thymidine with loss of water
414 Thymidine with loss of water with the addition of the matrix molecule
207 m/e 225 with the loss of an additional water molecule

DHB and sinipinic acid could not be used as several of the matrix ions overlap with the masses of the ions from thymidine.

As can be seen from the solution data the formation of the Thymidine ions can only be a result of the dissociation of the linker attached to the glass plate. Otherwise the trityl group would carry the charge and the thymidine would not be observed in positive ion mode. Use of other matrices may give better signal to noise and well as fewer ions in the same mass range as the Thymidine.

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties.

1. A method comprising:
(a) providing an array, the array comprising a substrate having a plurality of features, wherein at least one of the features of the array is a cleavable feature, the cleavable feature comprising one or more polynucleotides bound to the substrate via a cleavable linker;
(b) contacting the cleavable feature with a matrix material;
(c) analyzing the one or more polynucleotides using MALDI-MS to obtain information about the one or more polynucleotides; and
(d) evaluating a manufacturing process used to produce the array based on said information.

2. The method of claim 1, wherein evaluating includes determining a quality control measurement of the array.

3. The method of claim 1, wherein evaluating includes determining a quality control measurement of additional arrays contemporaneously manufactured with the array provided in step (a).

4. The method of claim 1, wherein providing an array comprises obtaining a multi-array substrate and dividing the multi-array substrate into individual arrays.

5. The method of claim 1, wherein providing an array comprises obtaining an array from the manufacturing process.

6. The method of claim 1, wherein evaluating includes providing feedback to the manufacturing process.

7. The method of claim 1, wherein analyzing the one or more polynucleotides comprises directing laser radiation at the matrix material to generate ions including ions derived from the one or more polynucleotides, and analyzing the ions in a mass spectrometer to obtain the information about the one or more polynucleotides.

8. The method of claim 1, wherein the cleavable linker is a triaryl methyl linker group.

9. The method of claim 8, wherein providing an array comprises synthesizing the polynucleotide on the substrate.

10. The method of claim 9, wherein synthesizing the polynucleotide on the substrate comprises providing a functionalized substrate having a nucleotide monomer bound to the substrate via the triaryl methyl linker group, and then synthesizing the polynucleotide using the nucleotide monomer bound to the substrate as a starting point for synthesizing the polynucleotide such that the resulting polynucleotide is bound to the substrate via the triaryl methyl linker group.

11. The method of claim 8, wherein providing an array comprises procuring the polynucleotide in solution and contacting the polynucleotide in solution with a functionalized substrate to result in the polynucleotide bound to the substrate via the triaryl methyl linker group.

12. The method of claim 8, wherein the triaryl methyl linker group is covalently bound to the polynucleotide directly or via an intermediate linking group.

13. The method of claim 8, wherein the triaryl methyl linker group has the structure (II) 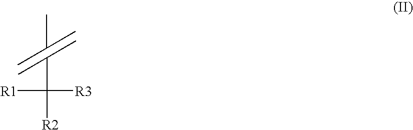
wherein the broken line represents a bond via which the triaryl methyl linker group is connected to the polynucleotide, and R1, R2, and R3 are independently selected from substituted or unsubstituted aryl groups, provided that one of R1, R2, and R3 is substituted by being bonded to the substrate.

14. The method of claim 13, wherein R1, R2, and R3 are independently selected from substituted phenyl and unsubstituted phenyl.

15. The method of claim 13, wherein R1, R2, and R3 are optionally substituted aryl groups independently selected from phenyl, biphenyl, naphthanyl, indolyl, pyridinyl, pyrrolyl, thiophenyl, furanyl, annulenyl, quinolinyl, and anthracenyl.

16. The method of claim 15, wherein at least one of R1, R2, and R3 is selected from naphthanyl, indolyl, pyridinyl, pyrrolyl, thiophenyl, furanyl, annulenyl, quinolinyl, and anthracenyl.

17. The method of claim 13, wherein R1, R2, and R3 are independently selected from phenyl, methoxyphenyl, dimethoxyphenyl, trimethoxyphenyl, and furanyl.

18. The method of claim 1, wherein the triaryl methyl linker group has the structure (II) 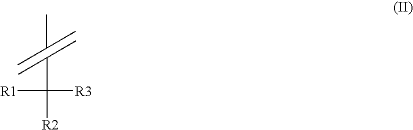
wherein the broken line represents a bond via which the triaryl methyl linker group is connected to the substrate, and R1, R2, and R3 are independently selected from substituted or unsubstituted aryl groups, provided that one of R1, R2, and R3 is substituted by being bonded to the polynucleotide.

19. The method of claim 18, wherein R1, R2, and R3 are independently selected from substituted phenyl and unsubstituted phenyl.

20. The method of claim 18, wherein R1, R2, and R3 are optionally substituted aryl groups independently selected from phenyl, biphenyl, naphthanyl, indolyl, pyridinyl, pyrrolyl, thiophenyl, furanyl, annulenyl, quinolinyl, and anthracenyl.

21. The method of claim 20, wherein at least one of R1, R2, and R3 is selected from naphthanyl, indolyl, pyridinyl, pyrrolyl, thiophenyl, furanyl, annulenyl, quinolinyl, and anthracenyl.

22. The method of claim 18, wherein R1, R2, and R3 are independently selected from phenyl, methoxyphenyl, dimethoxyphenyl, trimethoxyphenyl, and furanyl.

23. The method of claim 1, wherein the substrate is a mass spectrometer sample plate adapted to be disposed in operational relationship to a mass spectrometer to allow matrix assisted laser desorption/ionization analysis of the polynucleotide.
